(19) United States
(12) Patent Application Publication
(10) Pub. No.: US 2026/0357257 A1
(43) Pub. Date: Nov. 12, 2026

(54) NOVEL DOUBLE-STRANDED SIRNA, CONJUGATE THEREOF, AND USE THEREOF

(71) Applicant: SUNSHINE LAKE PHARMA CO., LTD., Dongguan, Guangdong (CN)

(72) Inventors: Qingyun REN, Dongguan (CN); Zhifu ZOU, Dongguan (CN); Yunfu CHEN, Dongguan (CN); Huimeng CHEN, Dongguan (CN); Pu WANG, Dongguan (CN); Yingjun ZHANG, Dongguan (CN)

(73) Assignee: SUNSHINE LAKE PHARMA CO., LTD., Dongguan, Guangdong (CN)

(21) Appl. No.: 19/487,321

(22) PCT Filed: May 31, 2024
(86) PCT No.: PCT/CN2024/096557

§ 371 Date: Nov. 24, 2025

(30) Foreign Application Priority Data
Jun. 1, 2023 (CN) 202310641202.3

(51) Int. Cl. C12N 15/113

(52) U.S. Cl. CPC C12N 15/113; C12N 2310/14; C12N 2310/315; C12N 2310/322; C12N 2310/351

(57) ABSTRACT

A novel double-stranded siRNA, conjugate thereof, and use thereof. A double-stranded siRNA, a conjugate thereof or a salt thereof, and also relates to use of double-stranded siRNA and the conjugate thereof in preparing a medicament for treating and/or preventing hepatitis B. The present invention also relates to a novel compound, and a use of the compound in raw materials for solid-phase synthesis of DNA nucleotides, raw materials for synthesis of siRNA drugs, siRNA drug research, gene function research and/or screening of whole gene libraries, the use of the compound as a raw material for synthesis of the double-stranded siRNA drug described in the present invention. The present invention also relates to a novel nucleotide residue and its use in siRNA drug research, as a raw material for the synthesis of gene function research, and/or as a raw material for screening of whole gene libraries, and an embedding group in oligonucleotides.

### FIELD OF THE INVENTION

The present invention belongs to the field of small nucleic acid drugs. The purpose of the present invention is to provide a novel double-stranded siRNA, a conjugate thereof and a use thereof. The present invention also provides use of the double-stranded siRNA and conjugate thereof in preparing a drug for treating and/or preventing hepatitis B. In addition, the present invention also relates to a novel compound, and a use of the compound in raw materials for solid-phase synthesis of DNA nucleotides, raw materials for synthesis of oligonucleotide drugs, raw materials for synthesis of siRNA drugs, siRNA drug research, gene function research and/or screening of whole gene libraries, especially the use of the compound as a raw material for synthesis of the double-stranded siRNA drug described in the present invention. Furthermore, the present invention also relates to a novel nucleotide residue and its use in siRNA drug research, as a raw material for the synthesis of gene function research, and/or as a raw material for screening of whole gene libraries, as well as an embedding group in oligonucleotides.

### BACKGROUND ART

Viral hepatitis B (abbreviated as hepatitis type B or hepatitis B) is a serious infectious disease that threatens the world, especially China. Currently, the two major types of hepatitis B treatment drugs recognized worldwide are interferon and nucleoside analogs. However, these two types of drugs have many disadvantages, such as easy development of drug resistance or limited use after use. For example, interferon is prone to adverse reactions, and nucleoside drugs have drug resistance and recurrence after discontinuation of the drug. Therefore, if the gene expression of the virus can be silenced at the genetic level, blocking the generation and replication of HBV, thereby fundamentally reducing viral replication and infection of liver cells, it will undoubtedly be the most ideal treatment for hepatitis B. Small interfering ribonucleic acid, i.e., small interfering RNA (siRNA) or short interfering RNA, can based on the RNA interference (RNAi) mechanism, inhibit or block the expression of any target gene of interest (such as genes that cause diseases like cancer) in a sequence-specific manner, thereby achieving therapeutic purposes.

RNA interference (RNAi) is a phenomenon in which double-stranded RNA (dsRNA) molecules shut down or silence the expression of homologous genes at the mRNA level. RNA interference technology, also known as gene knock-down or gene silencing, is a typical post-transcriptional gene regulation method, also known as post-transcriptional gene silencing (PTGS). The earliest report on RNA interference appeared in 1990, when two different research groups simultaneously reported the phenomenon of RNA interference in transgenic plants. Subsequently, RNA interference was observed in almost all eukaryotic organisms, including nematodes, fruit flies, zebrafish and mice. In 1999, Hamilton and Baulcombe detected RNA fragments of 21-25 nucleotides in length in plants undergoing RNA interference. These RNA fragments were shown to be necessary for RNA interference and were called small interfering nucleic acids (siRNAs). Double-stranded siRNA forms an RNA-induced silencing complex (RISC) with cell-derived related enzymes and proteins. During RNA interference, the sense strand in the double-stranded siRNA is excluded from the complex, and the antisense strand guides RISC to bind to the homologous site of the target mRNA. The target mRNA is then degraded by RNase III in the complex, thereby shutting down the expression of the target gene.

### SUMMARY

The present invention aims to provide a novel double-stranded siRNA, a conjugate thereof, a salt thereof and a use thereof, and specifically relates to the use of the double-stranded siRNA and conjugate thereof in preparing a drug for treating and/or preventing hepatitis B. In addition, the present invention also relates to a novel compound, and a use of the compound in raw materials for solid-phase synthesis of DNA nucleotides, raw materials for synthesis of siRNA drugs, siRNA drug research, gene function research and/or screening of whole gene libraries, especially the use of the compound as a raw material for synthesis of the double-stranded siRNA drug described in the present invention. Furthermore, the present invention also relates to a novel nucleotide residue and its use in siRNA drug research, as a raw material for the synthesis of gene function research, and/or as a raw material for screening of whole gene libraries, as well as an embedding group in oligonucleotides. The double-stranded siRNA and conjugate thereof of the present invention have higher gene expression inhibition activity and/or lower toxicity against HBV.

In one aspect, the present invention relates to a double-stranded siRNA, a conjugate thereof, or a salt thereof, wherein, the double-stranded siRNA comprises a sense strand and an antisense strand, and the double-stranded siRNA comprises one or more Y, the Y is

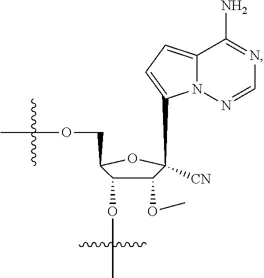 [SMILES: *OC[C@H]1O[C@@](C#N)(c2ccc3c(N)ncnn23)[C@H](OC)[C@@H]1O*]

wherein each of nucleotides in the double-stranded siRNA and Y is independently modified or unmodified. Optionally, the Y can be at any position in the double-stranded siRNA.

In some embodiments of the double-stranded siRNA, conjugate or salt thereof of the present invention, wherein, the double-stranded siRNA comprises 1, 2, 3, 4, 5, 6, 7, 8, 9 or 10 Y.

In some embodiments of the double-stranded siRNA, conjugate or salt thereof of the present invention, wherein, the double-stranded siRNA comprises 1, 2, 3, 4 or 5 Y.

In some embodiments of the double-stranded siRNA, conjugate or salt thereof of the present invention, wherein, the antisense strand comprises a sequence of 5′-UGUGAAGCGAAGUGCACACUU-3′ (SEQ ID NO:40), or a sequence in which one or more nucleotide residues are replaced by Y in the sequence of SEQ ID NO:40; wherein each of nucleotides and Y in the antisense strand is independently modified or unmodified, and the length of the antisense strand is not more than 23 nucleotides, wherein Y is as defined herein. Optionally, the Y substitution occurs at any position in the sequence of SEQ ID NO:40.

In some embodiments of the double-stranded siRNA, conjugate or salt thereof of the present invention, wherein, the sense strand comprises & sequence of 5′-GUGUGCACUUCGCUUCACA-3′ (SEQ ID NO:20), or a sequence in which one or more nucleotide residues are replaced by Y in the sequence of SEQ ID NO:20, wherein each of nucleotides and Y in the sense strand is independently modified or unmodified, and the length of the sense strand is not more than 23 nucleotides. Optionally, the Y substitution occurs at any position in the sequence of SEQ ID NO:20.

In some embodiments of the double-stranded siRNA, conjugate or salt thereof of the present invention, more than 70%, 75%, 80%, 85%, 90% or 95% of the nucleotides and Y in the sense strand or the antisense strand of the double-stranded siRNA are modified.

In some embodiments of the double-stranded siRNA, conjugate or salt thereof of the present invention, more than 70%, 75%, 80%, 85%, 90% or 95% of the nucleotides and Y in the sense strand and the antisense strand of the double-stranded siRNA are modified.

In some embodiments of the double-stranded siRNA, conjugate or salt thereof of the present invention, all the nucleotides and Y in the double-stranded siRNA are modified.

In some embodiments of the double-stranded siRNA, conjugate or salt thereof of the present invention, the modification is selected from at least one of the following: 2′-methoxy modification, 2′-methoxyethyl modification, 2′-fluoro modification, phosphorothioate linkage, 2′-deoxy modification, 2′-amino modification, locked nucleic acid modification, unlocked nucleic acid modification, ethylene glycol nucleic acid modification and 5′-vinyl phosphate ester modification.

In some embodiments of the double-stranded siRNA, conjugate or salt thereof of the present invention, wherein, the sense strand comprises a sequence in which 0, 1, 2, 3, 4 or 5 nucleotide residues are replaced with Y in the sequence of SEQ ID NO:20, wherein each of nucleotides and Y in the sense strand are independently modified or unmodified.

In some embodiments of the double-stranded siRNA, conjugate or salt thereof of the present invention, wherein, the sense strand comprises a sequence in which one nucleotide residue is replaced with Y in the sequence of SEQ ID NO:20.

In some embodiments of the double-stranded siRNA, conjugate or salt thereof of the present invention, wherein, the antisense strand comprises a sequence in which 0, 1, 2, 3, 4 or 5 nucleotide residues are replaced with Y in the sequence of SEQ ID NO:40, wherein each of nucleotides and Y in the antisense strand are independently modified or unmodified. In some embodiments of the double-stranded siRNA, conjugate or salt thereof of the present invention, wherein, the antisense strand comprises a sequence in which one nucleotide residue is replaced with Y in the sequence of SEQ ID NO:40.

In some embodiments of the double-stranded siRNA, conjugate or salt thereof of the present invention, wherein, the Y substitution occurs at positions 1 to 19 from the 5′ end of the sequence of SEQ ID NO: 20 (i.e., the Y substitution optionally occurs at position 1, 2, 3, 4, 5, 6, 7, 8, 9, 10, 11, 12, 13, 14, 15, 16, 17, 18 or 19 from the 5′ end of the sequence of SEQ ID NO: 20).

In some embodiments of the double-stranded siRNA, conjugate or salt thereof of the present invention, wherein, the Y substitution occurs at positions 1 to 21 from the 5′ end of the sequence of SEQ ID NO: 40 (i.e., the Y substitution optionally occurs at position 1, 2, 3, 4, 5, 6, 7, 8, 9, 10, 11, 12, 13, 14, 15, 16, 17, 18, 19, 20 or 21 from the 5′ end of the sequence of SEQ ID NO: 40).

In some embodiments of the double-stranded siRNA, conjugate or salt thereof of the present invention, wherein, the sense strand comprises a sequence in which 0, 1, 2, 3, 4 or 5 nucleotide residues are substituted with Y in the sequence of 5′-g•u•guGcACUucgcuucaca-3′ (SEQ ID NO: 19), wherein each of nucleotides and Y in the sense strand is independently modified or unmodified, and the Y substitution occurs at positions 1 to 19 from the 5′ end of the sequence of SEQ ID NO: 19 (i.e., the Y substitution optionally occurs at position 1, 2, 3, 4, 5, 6, 7, 8, 9, 10, 11, 12, 13, 14, 15, 16, 17, 18 or 19 form the 5′ end of the sequence of SEQ ID NO: 19), and the length of the sense strand is not more than 23 nucleotides.

In some embodiments of the double-stranded siRNA, conjugate or salt thereof of the present invention, wherein, the antisense strand comprises a sequence in which 0, 1, 2, 3, 4 or 5 nucleotide residues are substituted with Y in the sequence of 5′-u•G•ugaAgCGaaguGcAcac•u•u-3′ (SEQ ID NO: 36), wherein each of nucleotides and Y in the antisense strand is independently modified or unmodified, and the Y substitution occurs at positions 1 to 21 from the 5′ end of the antisense strand of SEQ ID NO: 36 (i.e., the Y substitution optionally occurs at position 1, 2, 3, 4, 5, 6, 7, 8, 9, 10, 11, 12, 13, 14, 15, 16, 17, 18 or 19 from the 5′ end of the sequence of SEQ ID NO: 36), and the length of the antisense strand is not more than 23 nucleotides.

In some embodiments of the double-stranded siRNA, conjugate or salt thereof of the present invention, wherein, the antisense strand comprises a sequence in which 0, 1, 2, 3, 4 or 5 nucleotide residues are substituted with Y in the sequence of 5′-u•G•uga(Agn)gCGaaguGcAcac•u•u-3′ (SEQ ID NO: 33), wherein each of nucleotides and Y in the antisense strand is independently modified or unmodified, and the Y substitution occurs at positions 1 to 21 from the 5′ end of the sequence of SEQ ID NO: 33 (i.e., the Y substitution optionally occurs at position 1, 2, 3, 4, 5, 6, 7, 8, 9, 10, 11, 12, 13, 14, 15, 16, 17, 18 or 19 from the 5′ end of the sequence of SEQ ID NO: 33), and the length of the antisense strand is not more than 23 nucleotides.

Unless otherwise specified, in the context of the present invention, capital letters C, G, U and A represent the base composition of natural nucleotides; lowercase letters c, g, u and a respectively represent that the 2-position of the ribose on the nucleotide represented by the corresponding capital letter is modified by methoxy; an underline under a capital letter represents that the 2-position of the ribose on the nucleotide represented by the letter is modified by fluorine; Agn is adenosine-ethylene glycol nucleic acid; the middle dot “•” represents that there is phosphorothioate linkage between two nucleotide residues adjacent to the left and right sides of the middle dot “•”; Y

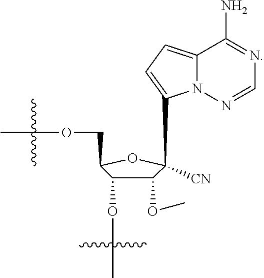 [SMILES: *OC[C@H]1O[C@@](C#N)(c2ccc3c(N)ncnn23)[C@H](OC)[C@@H]1O*]

In some embodiments of the double-stranded siRNA, conjugate or salt thereof of the present invention, the sense strand comprises one of the following nucleotide sequences:
SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:21;
the length of the sense strand is not more than 23 nucleotides.

In some embodiments of the double-stranded siRNA, conjugate or salt thereof of the present invention, the antisense strand comprises one of the following nucleotide sequences:
SEQ ID NO:22, SEQ ID NO:23, SEQ ID NO:24, SEQ ID NO:25, SEQ ID NO:26, SEQ ID NO: 27, SEQ ID NO:28, SEQ ID NO:29, SEQ ID NO:30, SEQ ID NO:31, SEQ ID NO:32, SEQ ID NO: 33, SEQ ID NO:34, SEQ ID NO:35, SEQ ID NO:36, SEQ ID NO:37, SEQ ID NO:38 and SEQ ID NO: 39;
the length of the antisense strand is not more than 23 nucleotides.

In some embodiments of the double-stranded siRNA, conjugate or salt thereof of the present invention, wherein, the double-stranded siRNA comprises one of the following:
A sense strand comprising the nucleotide sequence of SEQ ID NO: 15 and an antisense strand comprising the nucleotide sequence of SEQ ID NO: 25, wherein the sense strand and antisense strand are each independently not more than 23 nucleotides;
A sense strand comprising the nucleotide sequence of SEQ ID NO: 17 and an antisense strand comprising the nucleotide sequence of SEQ ID NO: 25, wherein the sense strand and antisense strand are each independently not more than 23 nucleotides;
A sense strand comprising the nucleotide sequence of SEQ ID NO: 15 and an antisense strand comprising the nucleotide sequence of SEQ ID NO: 26, wherein the sense strand and antisense strand are each independently not more than 23 nucleotides;
A sense strand comprising the nucleotide sequence of SEQ ID NO: 16 and an antisense strand comprising the nucleotide sequence of SEQ ID NO: 26, wherein the sense strand and antisense strand are each independently not more than 23 nucleotides;
A sense strand comprising the nucleotide sequence of SEQ ID NO: 17 and an antisense strand comprising the nucleotide sequence of SEQ ID NO: 26, wherein the sense strand and antisense strand are each independently not more than 23 nucleotides;
A sense strand comprising the nucleotide sequence of SEQ ID NO: 15 and an antisense strand comprising the nucleotide sequence of SEQ ID NO: 27, wherein the sense strand and antisense strand are each independently not more than 23 nucleotides;
A sense strand comprising the nucleotide sequence of SEQ ID NO: 16 and an antisense strand comprising the nucleotide sequence of SEQ ID NO: 27, wherein the sense strand and antisense strand are each independently not more than 23 nucleotides;
A sense strand comprising the nucleotide sequence of SEQ ID NO: 17 and an antisense strand comprising the nucleotide sequence of SEQ ID NO: 27, wherein the sense strand and antisense strand are each independently not more than 23 nucleotides;
A sense strand comprising the nucleotide sequence of SEQ ID NO: 16 and an antisense strand comprising the nucleotide sequence of SEQ ID NO: 28, wherein the sense strand and antisense strand are each independently not more than 23 nucleotides;
A sense strand comprising the nucleotide sequence of SEQ ID NO: 17 and an antisense strand comprising the nucleotide sequence of SEQ ID NO: 28, wherein the sense strand and antisense strand are each independently not more than 23 nucleotides; or
A sense strand comprising the nucleotide sequence of SEQ ID NO: 18 and an antisense strand comprising the nucleotide sequence of SEQ ID NO: 28, wherein the sense strand and antisense strand are each independently not more than 23 nucleotides.

In some embodiments of the double-stranded siRNA, conjugate or salt thereof of the present invention, the double-stranded siRNA or conjugate thereof is selected from one of S1 to S143, S146, S147 and S150 in Table 1 of the present specification. In some embodiments of the double-stranded siRNA, conjugate or salt thereof of the present invention, the double-stranded siRNA conjugate comprises the double-stranded siRNA and a conjugation group, that is, the double-stranded siRNA conjugate is a conjugate of the double-stranded siRNA conjugated to the conjugation group, wherein the conjugation group includes but is not limited to L-96 or DAW40007-4, and the structures of L-96 and DAW40007-4 are respectively:

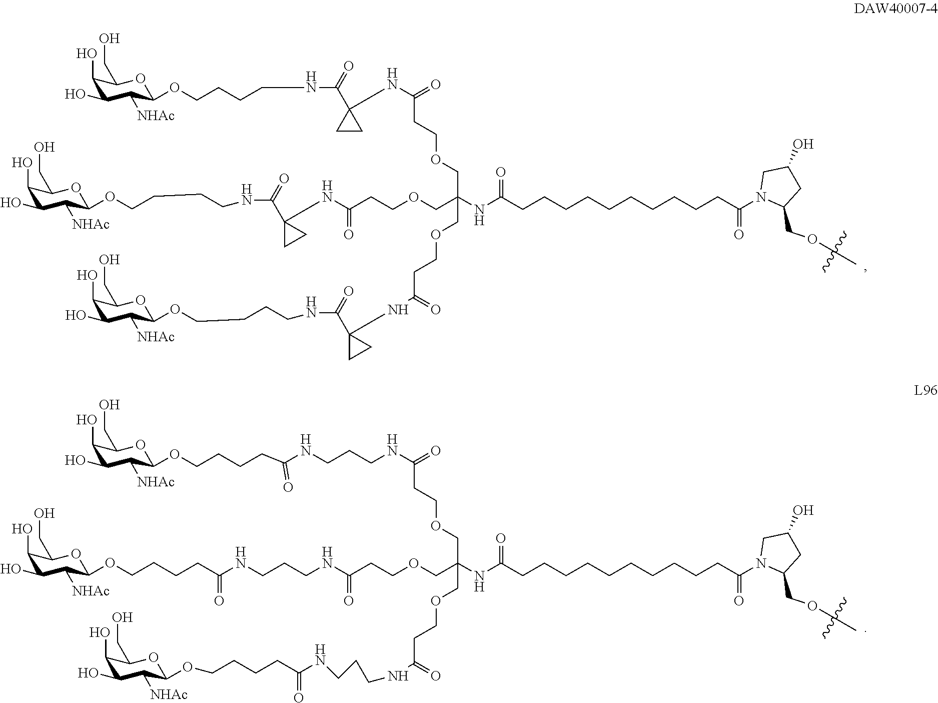

In some embodiments of the double-stranded siRNA, conjugate or salt thereof of the present invention, the conjugated group is conjugated to the 3′ end or the 5′ end of the sense strand of the double-stranded siRNA.

In some embodiments of the double-stranded siRNA, conjugate or salt thereof of the present invention, the conjugated group is conjugated to the 3′ end of the sense strand of the double-stranded siRNA.

In some embodiments of the double-stranded siRNA, conjugate or salt thereof of the present invention, the double-stranded siRNA is linked to the conjugated group through a phosphate eater group, phosphorothioate group or a phosphate group.

In another aspect, the present invention also relates to a nucleotide residue Y having structure

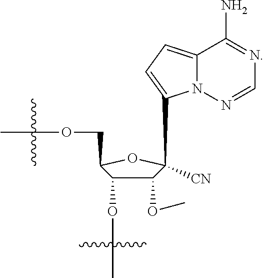 [SMILES: *OC[C@H]1O[C@@](C#N)(c2ccc3c(N)ncnn23)[C@H](OC)[C@@H]1O*]

In another aspect, the present invention also relates to a compound having Formula (I) or a stereoisomer, a tautomer or an acceptable salt of the compound having Formula (I),

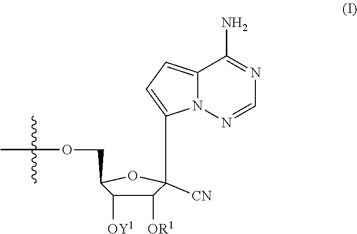

R¹ is methyl, ethyl, n-propyl, i-propyl, n-butyl, i-butyl or tert-butyl, each of the methyl, ethyl, n-propyl, i-propyl, n-butyl, i-butyl or tert-butyl is independently optionally substituted with 1, 2, 3 or 4 substituents selected from deuterium, hydroxyl, cyano, F, Cl, Br, I, methoxy, ethoxy, 1-propoxy and 2-propoxy;
Z and Y¹ are each independently H, deuterium, a hydroxyl protecting group, HOC(═O)(CH₂)ⱼC(═O)—, M-C(═O)(CH₂)ⱼC(═O)—, a phosphate ester group, a phosphorothioate group, a phosphoramidite group or a hydrogen phosphate group;
M is a solid support, preferably a hydroxyl- or amino-functionalized solid support, more preferably a resin or CPG, further preferably a macroporous resin or CPG, further preferably an aminomethyl resin, a hydroxyl resin or —NHCPG;
R is —NHR² or —N═CH—NR^{a}R^{b};
R² is an amino protecting group;
each of R^{a} and R^{b} is independently H or an amino protecting group, or R^{a}, R^{b} and the nitrogen atom to which they are attached form a heterocyclic group consisting of 5-6 ring atoms, each of heterocyclic group consisting of 5-6 ring atoms is independently optionally substituted with 1, 2, 3 or 4 substituents selected from deuterium, hydroxyl, cyano, F, Cl, Br, I, methyl, ethyl, n-propyl, isopropyl, methoxy, ethoxy, 1-propoxy and 2-propoxy;
wherein, each j is independently 1, 2, 3, 4 or 5.

In some embodiments of the compounds described herein, Z and Y′ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₁₂ alkyl, C₁₋₁₂ alkylC(═O)—, C₁₋₁₂ alkylsilyl, C₆₋₁₀ arylC₁₋₆ alkyl, HOC(═O)(CH₂)ⱼC(═O)—, M-C(═O)(CH₂)ⱼC(═O)—, triphenylmethyl, MMTr, DMTr, 4′,4′,4′-trimethoxytrityl,

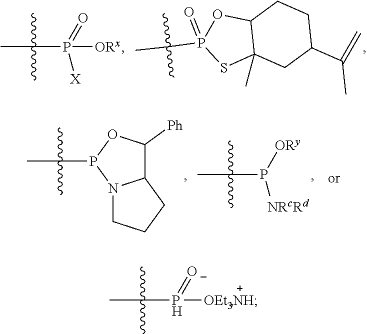

wherein, each j, M, X, R^{x}, R^{y}, R^{c} and R^{d} are as defined herein.

In some embodiments of the compounds described herein, Z and Y¹ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₁₀ alkyl, C₁₋₁₀ alkylC(═O)—, C₁₋₁₀ alkylsilyl, C₆₋₁₀ arylC₁₋₄ alkyl, HOC(═O)(CH₂)ⱼC(═O)—, M-C(═O)(CH₂)ⱼC(═O)—, triphenylmethyl, MMTr, DMTr, 4′,4′,4′-trimethoxytrityl,

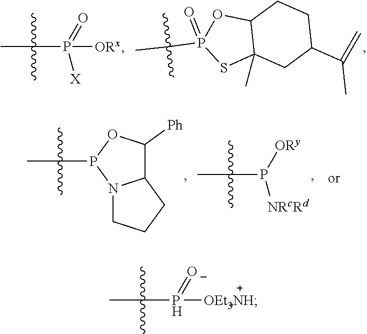

wherein, each j, M, X, R^{x}, R^{y}, R^{c} and R^{d} are as defined herein.

In some embodiments of the compounds described herein, Z and Y¹ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₈ alkyl, C₁₋₈ alkylC(═O)—, C₁₋₈ alkylsilyl, phenylC₁₋₃ alkyl, HOC(═O)(CH₂)ⱼC(═O)—, M-C(═O)(CH₂)ⱼC(═O)—, triphenylmethyl, MMTr, DMTr, 4′,4′,4′-trimethoxytrityl,

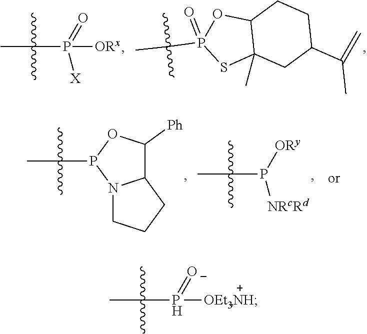

wherein, each j, M, X, R^{y}, R^{y}, R^{c} and R^{d} are as defined herein.

In some embodiments of the compounds described herein, Z and Y¹ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₆ alkyl, Cie alkylC(═O)—, C₁₋₆ alkylsilyl, benzyl, phenethyl, triphenylmethyl, MMTr, DMTr, 4′,4′,4′-trimethoxytrityl,

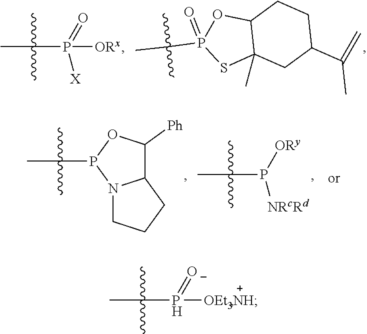

wherein, each j, M, X, R^{x}, R^{y}, R^{c} and R^{d} are as defined herein.

In some embodiments of the compounds described herein, X is Cl or Br.

In some embodiments of the compounds described herein, each of R^{x} and R^{y} is independently a hydroxy protecting group.

In some embodiments of the compounds described herein, each of R^{c} and R^{d} is independently H or an amino protecting group; or R^{c}, R^{d} and the nitrogen atom to which they are attached form a heterocyclic group consisting of 5-6 ring atoms, the heterocyclic group consisting of 5-6 ring atoms is independently optionally substituted with 1, 2, 3 or 4 substituents selected from deuterium, hydroxyl, cyano, F, Cl, Br, I, methyl, ethyl, propyl, methoxy, ethoxy, 1-propoxy and 2-propoxy.

In some embodiments of the compounds described herein, R² is 9-fluorenylmethoxycarbonyl, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, methyl, tert-butyl, 2-trimethylsilylethyl, 1,1-dimethyl-2-propenyl, 3-methyl-3-butenyl, allyl, benzyl, p-methoxybenzyldiphenylmethyl, trityl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, trimethylsilyl, triethylsilyl, triisopropylsilyl, C₁₋₆ alkoxy or R³C(═O)—, wherein, R³ is as defined herein.

In some embodiments of the compounds described herein, each of R^{a} and R^{b} is independently H, methyl, ethyl, n-propyl, i-propyl, n-butyl, i-butyl, tert-butyl, methoxy, ethoxy, 1-propoxy, 2-propoxy, 9-fluorenylmethoxycarbonyl, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2-trimethylsilylethyl, 1,1-dimethyl-2-propenyl, 3-methyl-3-butenyl, allyl, benzyl, p-methoxybenzyldiphenylmethyl, trityl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, trimethylsilyl, triethylsilyl, triisopropylsilyl or R⁴C(═O)—, or R^{a}, R^{b} and the N atom to which they are attached form a pyrrolidinyl, morpholinyl, piperidinyl or piperazinyl, each of pyrrolidinyl, morpholinyl, piperidinyl and piperazinyl is independently optionally substituted with 1, 2, 3 or 4 substituents selected from deuterium, hydroxyl, cyano, F, Cl, Br, I, methoxy, ethoxy, 1-propoxy and 2-propoxy, wherein, R⁴ is as defined herein.

In some embodiments of the compounds described herein, each of R³ and R⁴ is independently C₁₋₆ alkyl, halo C₁₋₆ alkyl, C₁₋₆ alkoxy, phenyl, halo-substituted phenyl, C₁₋₆ alkylphenyl or benzyl. In some embodiments of the compounds described herein, each of R³ and R⁴ is independently C₁₋₄ alkyl, halo C₁₋₄ alkyl, C₁₋₄ alkoxy, phenyl, halo-substituted phenyl, C₁₋₄ alkylphenyl or benzyl.

In some embodiments of the compounds described herein, each of R³ and R⁴ is independently methyl, ethyl, n-propyl, isopropyl, trifluoromethyl, difluoromethyl, monofluoromethyl, trichloromethyl, dichloromethyl, monochloromethyl, 2,2,2-trichloroethyl, methoxy, ethoxy, 1-propoxy, 2-propoxy, 1-butoxy, 2-methyl-1-propoxy, 2-butoxy, tert-butoxy, phenyl, halogen-substituted phenyl, C₁₋₃ alkylphenyl or benzyl.

In some embodiments of the compounds described herein, each of R^{x} and R^{y} is independently benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, cyano C₁₋₆ alkyl, C₁₋₆ alkyl, C₁₋₆ alkyl C(═O)—, C₁₋₆ alkylsilyl, phenyl, halogen-substituted phenyl, benzyl or phenethyl.

In some embodiments of the compounds described herein, each of R^{x} and R^{y} is independently benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, CNCH₂—, CN(CH₂)₂—, CNCH(CH₃)₂CH₂—, CN(CH₂)₃—, CNCH(CH₃)₂CH₂CH₂—, methyl, ethyl, n-propyl, i-propyl, n-butyl, i-butyl, tert-butyl, C₁₋₄ alkyl C(═O)—, C₁₋₄ alkylsilyl, phenyl, halogen-substituted phenyl, benzyl or phenethyl.

In some embodiments of the compounds described herein, each of R^{c} and R^{d} is independently methyl, ethyl, n-propyl, i-propyl, n-butyl, i-butyl, tert-butyl, methoxy, ethoxy, 1-propoxy, 2-propoxy, 9-fluorenylmethoxycarbonyl, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2-trimethylsilylethyl, 1,1-dimethyl-2-propenyl, 3-methyl-3-butenyl, allyl, benzyl, p-methoxybenzyldiphenylmethyl, trityl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, trimethylsilyl, triethylsilyl, triisopropylsilyl or R⁴C(═O)—, or R^{c}, R^{d} and the N atom to which they are attached form a pyrrolidinyl, morpholinyl, piperidinyl or piperazinyl, each of pyrrolidinyl, morpholinyl, piperidinyl and piperazinyl is independently optionally substituted with 1, 2, 3 or 4 substituents selected from deuterium, hydroxyl, cyano, F, Cl, Br, I, methoxy, ethoxy, 1-propoxy and 2-propoxy.

In some embodiments of the compounds described herein, the compound of the present invention has a structure as shown in Formula (II),

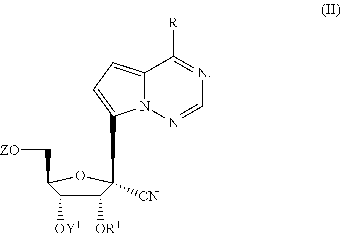

In another aspect of the present invention, the present invention also relates to a pharmaceutical composition comprising the double-stranded siRNA, conjugate or salt thereof according to the present invention, and a pharmaceutically acceptable carrier.

In another aspect of the present invention, the present invention also relates to the use of the double-stranded siRNA conjugate, conjugate thereof and the pharmaceutical composition of the present invention in the preparation of drugs for treating and/or preventing hepatitis B disease.

In another aspect of the present invention, the present invention also relates to the use of the nucleotide residues of the present invention in drug research, gene function research and/or screening of whole gene libraries for siRNA, and the structure of the nucleotide residues is:

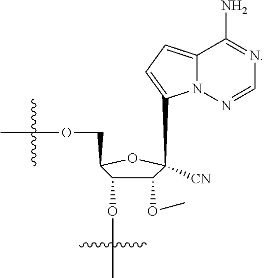 [SMILES: *OC[C@H]1O[C@@](C#N)(c2ccc3c(N)ncnn23)[C@H](OC)[C@@H]1O*]

In some embodiments of the application of the nucleotide residues described in the present invention, the siRNA drug is the double-stranded siRNA drug described in the present invention.

In another aspect of the present invention, the present invention also relates to a use of the compound in raw materials for solid-phase synthesis of DNA nucleotides, raw materials for synthesis of oligonucleotide drugs, raw materials for synthesis of siRNA drugs, siRNA drug research, gene function research and/or screening of whole gene libraries.

In another aspect of the present invention, the present invention also relates to the use of the nucleotide residues described in the present invention as oligonucleotide embedding groups, wherein the oligonucleotide is a nucleotide sequence containing 10 to 50 nucleotides or nucleotide base pairs, and the oligonucleotide can inhibit or block gene expression, and the structure of the nucleotide residue is:

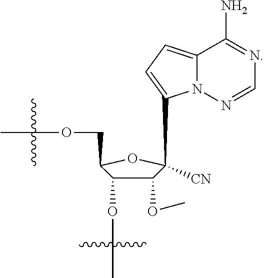 [SMILES: *OC[C@H]1O[C@@](C#N)(c2ccc3c(N)ncnn23)[C@H](OC)[C@@H]1O*]

In some embodiments of the use of the nucleotide residue as an oligonucleotide embedding group in the present invention, wherein, the gene is the HBV gene.

In some embodiments of the use of the nucleotide residue as an oligonucleotide embedding group in the present invention, wherein, the oligonucleotide is siRNA.

In some embodiments of the use of the nucleotide residue as an oligonucleotide embedding group in the present invention, wherein, the siRNA comprises the sense strand and the antisense strand.

In some embodiments of the use of the nucleotide residue as an oligonucleotide embedding group in the present invention, wherein, the nucleotide residue is embedded only into the sense strand of the siRNA.

In some embodiments of the use of the nucleotide residue as an oligonucleotide embedding group in the present invention, wherein, the nucleotide residue is embedded only into the antisense strand of the siRNA.

In some embodiments of the use of the nucleotide residue as an oligonucleotide embedding group in the present invention, wherein, the nucleotide residue is embedded into the sense strand and the antisense strand of the siRNA.

In some embodiments of the use of the nucleotide residue as an oligonucleotide embedding group in the present invention, wherein, the sense strand of the siRNA comprises the sequence of SEQ ID NO: 19.

In some embodiments of the use of the nucleotide residue as an oligonucleotide embedding group in the present invention, wherein, the antisense strand of the siRNA comprises the sequence of SEQ ID NO: 36 or SEQ ID NO: 33.

### DETAILED DESCRIPTION OF THE INVENTION

### Definitions and General Terminology

In the present invention, the term “comprise” or “include” is an open expression, it means comprising the contents disclosed herein, but don't exclude other contents.

In the present invention, the term “small interfering RNA” or “siRNA” refers to a type of double-stranded RNA molecule with a length of 17 to 30 nucleotides, comprising a sense strand and an antisense strand. siRNA mediates targeted cleavage of RNA transcripts through the RISC pathway by forming the RNA-induced silencing complex (RISC). Specifically, siRNA directs the specific degradation of mRNA sequences through a process known as RNA interference (RNAi), inhibiting the translation of mRNA into amino acids and protein. For example, siRNA can modulate (e.g., inhibit) the expression of hepatitis B virus in cells.

In the present invention, the term “antisense strand (or guide strand)” refers to the siRNA strand that includes a region that is substantially complementary to a target sequence (eg, a sequence in hepatitis B virus mRNA). The term “sense strand (or follower strand)” refers to the siRNA strand that contains a region that is substantially complementary to the antisense strand. The antisense strand or the sense strand in the double-stranded siRNA of the present invention contains one or more (eg, 2, 3, 4 or 5) nucleotide residues Y, and the Y has the structure of the present invention.

The term “substantially complementary” refers to complete complementarity or at least partial complementarity, for example, the antisense strand is completely complementary or at least partially complementary to the target sequence. In the case of partial complementarity, mismatches can occur within internal or terminal regions of the molecule, with the most tolerated mismatches occurring within the terminal regions, e.g., within 5, 4, 3, or 2 nucleotides of the 5′- and/or 3′-end of either strand of the siRNA.

It should be noted that “at least a portion of the antisense strand is substantially complementary to the mRNA” means that the antisense strand has a polynucleotide that is substantially complementary to a continuous portion of the mRNA of interest (eg, mRNA encoding hepatitis B virus). Alternatively, if a polynucleotide is substantially non-interruptedly complementary to a portion of an mRNA encoding a hepatitis B virus, then the antisense strand is complementary to at least a portion of the mRNA encoding the hepatitis B virus.

In the present invention, the term “target sequence” refers to a continuous portion of the nucleotide sequence of an mRNA molecule formed during transcription of a gene encoding hepatitis B virus, including mRNA that is an RNA processing product of a primary transcription product.

In the present disclosure, a double-stranded siRNA analogue refers to a complex of ribonucleic acid molecules. It has a double-stranded structure, comprises two antiparallel and substantially complementary nucleic acid strands, and has “sense” and “antisense” orientations relative to a target RNA. In the present disclosure, “complementary” has the meaning well known to those skilled in the art. That is, in a double-stranded nucleic acid molecule, bases of one strand pair with bases on the other strand in a complementary manner. A purine base adenine (A) is always paired with a pyrimidine base uracil (U); a pyrimidine base cytosine (G) is always paired with a purine base guanine (C). Each base pair comprises a purine and a pyrimidine. When adenines on one strand are always paired with uracils on the other strand, and guanines are always paired with cytosines, the two strands are considered complementary to each other, and the sequence of the strand can be deduced from the sequence of its complementary strand.

In the present invention, the term “inhibiting the expression of hepatitis B virus genes” includes any level of inhibition of hepatitis B virus (HBV) genes, for example, at least partial inhibition of HBV gene expression, such as inhibition of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%. The expression of the HBV gene can be evaluated based on the level of any variable related to the expression of the HBV gene, for example, the level of HBV mRNA or the level of HBV protein. Inhibition can be assessed by a decrease in the absolute or relative level of one or more of these variables compared to a control level. The control level can be any type of control level utilized in the art, e.g., a baseline level prior to administration, or a level determined from a similar subject, cell, or sample that has not been treated or has been treated with a control (e.g., a buffer-only control or a no active agent control).

In the present invention, “pharmaceutical composition” may refer to a composition used for treating a disease or for in vitro cell culture experiments. When used in the treatment of disease, the term “pharmaceutical composition” generally refers to unit dosage form and can be prepared by any of the methods well known in the art of pharmacy. All methods include the step of bringing the active ingredient into association with the excipient which constitutes one or more accessory ingredients. Typically, the composition is prepared by uniformly and thoroughly combining active double-stranded siRNA with a liquid excipient, a finely divided solid excipient, or both.

The siRNA of the present invention contains a nucleotide group as a basic structural unit. It is well known to those skilled in the art that the nucleotide group contains a phosphate group, a ribose group and a base, which will not be described in detail here. Each nucleotide in the siRNA is independently a modified or unmodified nucleotide, preferably an unmodified nucleotide. The double-stranded siRNA of the present invention contains a sense strand and an antisense strand.

The term “embed” as used herein means that an embedding group is linked to at least one nucleotide residue in the sequence, including replacing a nucleotide residue in the sequence with an embedding group (such as Y).

The “embedding group” described in the present invention is a residue of an analog of a natural nucleotide base, which is different from any natural nucleotide base disclosed in any patent. After being introduced into a nucleic acid sequence, it can give the sequence certain functions (such as bringing unexpected activities). As described in the present invention, Y, when embedded in an oligonucleotide sequence as a embedding group, can inhibit gene expression and thereby produce unexpected activities.

The “oligonucleotide embedded group” of the present invention refers to the embedding group connected to at least one nucleotide residue in the oligonucleotide, including replacing a nucleotide residue in the oligonucleotide with an embedding group (such as Y).

The Y-embedded sequence of the present invention refers to a sequence in which at least one nucleotide residue is connected to Y, including a sequence in which a nucleotide residue is replaced by Y. The Y-embedded sequence of the present invention may also be optionally modified, such as methoxy modification, fluorination modification, and phosphorothioate linkage. The Y-embedded sequence of the present invention includes but is not limited to: Y-embedded siRNA, Y-embedded positive strand and Y-embedded antisense strand.

The conjugate group of the present invention includes a pharmaceutically acceptable conjugate group. Generally speaking, the pharmaceutically acceptable conjugate group comprises a pharmaceutically acceptable targeting molecule and an optional linker. Exemplary conjugate groups, linkers, and targeting molecules can be found in WO2015006740A2 and CN114555188A. Exemplary conjugated groups include, but are not limited to, L96, NAG37, or DAW40007-4 described herein.

Unless otherwise indicated, “conjugation” refers to the covalent linkage of two or more chemical moieties, each with a specific function, to each other; accordingly, “conjugate” refers to a compound formed by covalent linkage of the chemical moieties.

The double-stranded siRNA conjugate of the present invention is a compound formed by connecting the double-stranded siRNA and a pharmaceutically acceptable conjugated group, and the double-stranded siRNA and the pharmaceutically acceptable conjugated group are covalently linked.

In the present invention, the term “pharmaceutically acceptable” means that the substance or composition must be chemically and/or toxicologically compatible with the other ingredients comprising the formulation and/or the mammal to be treated therewith. Preferably, the term “pharmaceutically acceptable” as used herein means approved by a regulatory agency of the Federal or a state government or listed in the U.S. Pharmacopeia or other generally recognized pharmacopeia for use in animals, and more particularly in humans.

In the present invention, the term “pharmaceutically acceptable excipients” may include any solvents, solid excipients, diluents or other liquid excipients, etc., which are suitable for a specific target dosage form. Except to the extent that any conventional excipients are incompatible with the double-stranded siRNA or its conjugate or salt thereof of the present invention, such as any adverse biological effects produced or interactions with any other components of the pharmaceutically acceptable composition in a deleterious manner, their use is also contemplated by the present invention.

As used herein, the term “treat” refers to administering a drug for the purpose of obtaining a desired pharmacological and/or physiological effect. The effect may be prophylactic in terms of complete or partial prevention of the disease or its symptoms, and/or therapeutic in terms of partial or complete cure of the disease and/or its adverse effects. As used herein, “treat” encompasses diseases in mammals, particularly humans, and includes: (a) preventing the occurrence of a disease or condition in an individual who is susceptible to the disease but has not yet been diagnosed with the disease; (b) inhibiting the disease, such as arresting the progression of the disease; or (c) alleviating the disease, such as alleviating the symptoms associated with the disease. As used herein, “treat” encompasses any administration of a drug, RNAi agent, or siRNA to a subject to treat, cure, alleviate, ameliorate, mitigate, or inhibit a disease in the subject, including but not limited to administering a drug containing an RNAi agent, siRNA, or siRNA conjugate described herein to a subject in need thereof.

Unless otherwise specified, in the context of the present invention, capital letters C, G, U and A represent the base composition of natural nucleotides; lowercase letters c, g, u and a respectively represent that the 2-position of the ribose on the nucleotide represented by the corresponding capital letter is modified by methoxy; an underline under a capital letter represents that the 2-position of the ribose on the nucleotide represented by the letter is modified by fluorine; the middle dot “•” represents that there is phosphorothioate linkage

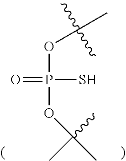

between two nucleotide residues adjacent to the left and right sides of the middle dot “•”, If there is no “•” between nucleotide residues, it means that the nucleotide residues are linked by phosphorothioate

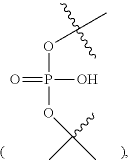

for example, “g•u” means that g and u residues are linked by phosphorothioate, and “gu” means that g and u residues are linked by phosphate groups; Agn is adenosine-glycol nucleic acid (Glycol nucleic acid, GNA,

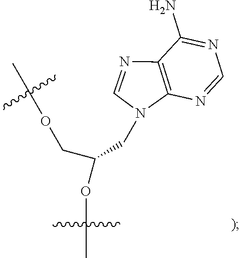

Y in double-stranded siRNA represents

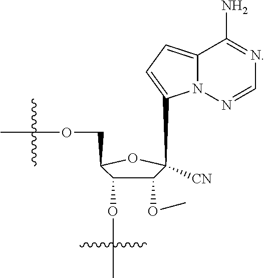

Wherein, the fluorine modification of the ribose position 2 in the present invention means that the hydroxyl group at the ribose position 2 is replaced by F; the methoxy modification of the ribose position 2 means that the hydroxyl group at the ribose position 2 is replaced by a methoxy group.

In the context of the present invention, Bz represents benzoyl; MMTr represents 4′-methoxytrityl; DMTr represents 4′,4′-dimethoxytriphenylmethyl.

In the present invention, “phosphate group”, “phosphate group” and “phosphate bond” are used interchangeably and include phosphate monoester group, phosphate diester group or phosphate triester group. The “phosphate group” in the “phosphorothioate group” has the same meaning. Unless otherwise specified, natural internucleotide phosphate groups are phosphodiester groups. Unless otherwise specified, the sequence in which Y replaces one or more nucleotide residues in the present invention means that one Y replaces one nucleotide residue, rather than one Y replacing two or more nucleotide residues. If two nucleotide residues are replaced, two Ys should replace two nucleotide residues respectively. For example, if the antisense strand comprises a sequence in which Y replaces zero, one, two, three, four, or five nucleotide residues in the sequence set forth in SEQ ID NO: 40, then it refers to a sequence in which 0, 1, 2, 3, 4 or 5 nucleotide residues in the sequence set forth in SEQ ID NO:40 are replaced by 0, 1, 2, 3, 4 or 5 Y, respectively.

The term “oxo” refers to a ═O group. For example, a carbon atom is linked to an oxygen atom via a double bond, whereby a ketone or an aldehyde is formed.

As used herein, “chemically modified” or “modification” means a structure that is chemically different when compared to a naturally occurring counterpart, including all changes by chemical means, such as the addition or removal of a chemical moiety, or the substitution of one chemical moiety for another.

The compounds of the present invention may be asymmetric, ie, have one or more stereoisomers. Unless otherwise indicated, all stereoisomers are included, as are enantiomers and diastereomers. The compounds of the present invention containing an asymmetric carbon atom can be isolated in optically pure or racemic forms. Optically pure forms can be resolved from racemic mixtures or synthesized by using chiral starting materials or reagents.

Optically active (R)- and(S)-isomers, as well as D and L isomers, can be prepared by chiral synthesis or chiral reagents or other conventional techniques. An enantiomer of a certain compound of the present disclosure can be prepared by asymmetric synthesis or derivatization using a chiral auxiliary, wherein the resulting diastereoisomeric mixture is separated and the auxiliary group is cleaved so as to provide the desired pure enantiomer. Alternatively, when the molecule contains a basic functional group (such as amino) or an acidic functional group (such as carboxyl), the compound reacts with an appropriate optically active acid or base to form a salt of the diastereoisomer, which is then subjected to diastereoisomeric resolution through conventional methods in the art followed by recovery to give the pure enantiomer. Furthermore, the enantiomer and the diastereoisomer are generally isolated through chromatography using a chiral stationary phase, optionally in combination with chemical derivatization (e.g., carbamate generated from amines).

The present invention also includes isotopically-labeled compounds of the invention which are identical to those recited herein, except that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁵Cl, respectively.

Unless otherwise indicated, when a position is specifically designated as deuterium (D), that position is understood to have an abundance of deuterium at least 1000 times greater than the natural abundance of deuterium (which is 0.015%) (i.e., at least 10% deuterium incorporation). Example compounds having an abundance greater than the natural abundance of deuterium can include at least 1000 times greater deuterium, at least 2000 times greater deuterium, at least 3000 times greater deuterium, at least 4000 times greater deuterium, at least 5000 times greater deuterium, at least 6000 times greater deuterium, or even greater abundances of deuterium. The present invention also encompasses the compounds of formula (I) in various deuterated forms. Each available hydrogen atom attached to a carbon atom can independently be replaced with a deuterium atom. Those skilled in the art can synthesize the deuterated form of the compound of formula (I) by referring to relevant literature. In preparing deuterated forms of compounds of formula (I), commercially available deuterated starting materials may be used, or they may be synthesized using conventional techniques using deuterated reagents, including but not limited to deuterated borane, trideuterated borane in tetrahydrofuran, deuterated lithium aluminum hydride, deuterated iodoethane, deuterated iodomethane, and the like.

The conjugated groups described herein can enhance the delivery of therapeutic agents to specific target locations (eg, specific organs or tissues) within a subject, such as a human or animal. In some embodiments of the present invention, the conjugated group can enhance the targeted delivery of expression-inhibitory double-stranded siRNA. In some embodiments of the present invention, the conjugated group can enhance the targeted delivery of expression-inhibitory double-stranded siRNA to the liver.

The conjugate groups described herein can be linked directly or indirectly to a compound, such as a therapeutic agent, e.g., an expression inhibitory oligonucleotide, e.g., to the 3′ or 5′ end of an expression inhibitory oligonucleotide. In some embodiments of the invention, the expression inhibitory oligonucleotide comprises one or more modified nucleotides. In some embodiments of the invention, the expression-inhibiting oligonucleotide is an RNAi agent, such as a double-stranded siRNA agent comprising a sense strand and an antisense strand. In some embodiments of the present invention, the conjugated group disclosed herein is attached to the 3′ end of the sense strand of a double-stranded siRNA agent. In some embodiments, the conjugate group disclosed herein is linked to the expression inhibitory oligonucleotide agent at the 3′ end of the sense strand of the double-stranded siRNA agent via a phosphate, phosphorothioate, or phosphonate group.

Stereochemical definitions and conventions used herein generally follow S. P. Parker Ed., McGraw-Hill Dictionary of Chemical Terms (1984) McGraw-Hill Book Company, New York and Eliel et al., “Stereochemistry of Organic Compounds”, John Wiley & Sons, Inc., New York, 1994. The compounds disclosed herein may contain asymmetric or chiral centers, and therefore exist in different stereoisomeric forms. It is intended that all stereoisomeric forms of the compounds disclosed herein, including, but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and/or (+) and (−) are employed to designate the sign of rotation of plane-polarized light by the compound, with (−) or/meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The term “racemic mixture” or “racemate” refers to an equimolar mixture of two enantiomeric species, devoid of optical activity.

The term “tautomer” or “tautomeric form” refers to structural isomers of different energies which are interconvertible via a low energy barrier. Some non-limiting examples of proton tautomers (also known as prototropic tautomers) include interconversions via migration of a proton, such as keto-enol and imine-enamine isomerizations.

The term “composition” refers to a mixture of a drug containing one or more compounds described herein, or a physiologically acceptable salt or precursor thereof, and other chemical components, as well as other components such as physiologically acceptable carriers and excipients. The purpose of the composition is to facilitate administration to an organism, facilitate the absorption of the active ingredient and thus exert biological activity.

The term “pharmaceutically acceptable excipient” or “pharmaceutically acceptable excipient” includes, but is not limited to, any adjuvant, carrier, excipient, glidant, sweetener, diluent, preservative, dye/colorant, flavoring agent, surfactant, wetting agent, dispersant, suspending agent, stabilizer, isotonic agent, solvent or emulsifier that has been approved by the U.S. Food and Drug Administration as acceptable for use in humans or domestic animals.

Unless otherwise specified, the “compound”, “ligand”, “nucleic acid conjugate”, “double-stranded siRNA conjugate”, “double-stranded siRNA” and “nucleic acid” of the present invention can independently exist in the form of salt, mixed salt or non-salt (such as free acid or free base). When present in the form of a salt or mixed salt, it may be a pharmaceutically acceptable salt.

The term “acceptable salts” includes acceptable acid addition salts and pharmaceutically acceptable base addition salts. “Acceptable acid addition salts” refer to salts formed with inorganic or organic acids that retain the biological effectiveness of the free base without other adverse effects. Inorganic acid salts include, but are not limited to, hydrochloride, hydrobromide, sulfate, nitrate, phosphate, and the like; organic acid salts include, but are not limited to, formate, acetate, 2,2-dichloroacetate, trifluoroacetate, propionate, caproate, octanoate, decanoate, undecylenate, glycolate, gluconate, lactate, sebacate, adipate, glutarate, malonate, oxalate, maleate, succinate, fumarate, tartrate, citrate, palmitate, stearate, oleate, cinnamate, laurate, malate, glutamate, pyroglutamate, aspartate, benzoate, methanesulfonate, benzenesulfonate, p-toluenesulfonate, alginate, ascorbate, salicylate, 4-aminosalicylate, naphthalene disulfonate, and the like. These salts can be prepared by methods known in the art.

“Pharmaceutically acceptable base addition salt” refers to a salt formed with an inorganic base or an organic base that retains the biological effectiveness of the free acid without other side effects. Salts derived from inorganic bases include, but are not limited to, sodium, potassium, lithium, ammonium, calcium, magnesium, iron, zinc, copper, manganese, aluminum salts, and the like. Preferred inorganic salts are ammonium salts, sodium salts, potassium salts, calcium salts and magnesium salts, preferably sodium salts. Salts derived from organic bases include, but are not limited to, salts of primary, secondary, and tertiary amines, substituted amines, including naturally substituted amines, cyclic amines, and basic ion exchange resins, such as ammonia, isopropylamine, trimethylamine, diethylamine, triethylamine, tripropylamine, ethanolamine, diethanolamine, triethanolamine, dimethylethanolamine, 2-dimethylaminoethanol, 2-diethylaminocthanol, dicyclohexylamine, lysine, arginine, histidine, caffeine, procaine, choline, betaine, ethylenediamine, glucosamine, methylglucamine, theobromine, purines, piperazine, piperidine, N-ethylpiperidine, polyamine resins, and the like. Preferred organic bases include isopropylamine, diethylamine, ethanolamine, trimethylamine, dicyclohexylamine, choline and caffeine. These salts can be prepared by methods known in the art.

As described herein, compounds disclosed herein may optionally be substituted with one or more substituents, such as are illustrated generally below, or as exemplified by particular classes, subclasses, and species of the invention. In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position.

Furthermore, unless otherwise stated, the phrase “each . . . is independently” is used interchangeably with the phrase “each (of) . . . and . . . is independently”. It should be understood broadly that the specific options expressed by the same symbol are independently of each other in different radicals; or the specific options expressed by the same symbol are independently of each other in same radicals.

In the present invention, the terms “optionally” or “optional” generally mean that the subsequently described event or circumstance can but need not occur, and that the description includes instances where the event or circumstance occurs and instances where it does not occur.

At various places in the present specification, substituents of compounds disclosed herein are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl and C₆ alkyl.

The term “alkyl” or “alkyl group” refers to a saturated linear or branched-chain monovalent hydrocarbon radical of 1 to 20 carbon atoms, wherein the alkyl radical may be optionally and independently substituted with one or more substituents described herein. In some embodiments, the alkyl group contains 1-12 carbon atoms. In other embodiments, the alkyl group contains 1-10 carbon atoms. In other embodiments, the alkyl group contains 1-8 carbon atoms. In still other embodiments, the alkyl group contains 1-6 carbon atoms. In yet other embodiments, the alkyl group contains 1~4 carbon atoms and in still yet other embodiments, the alkyl group contains 1-3 carbon atoms. Some non-limiting examples of the alkyl group include, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), 2-methylpropyl or isobutyl (i-Bu, —CH₂CH(CH₃)₂), 1-methylpropyl or sec-butyl (s-Bu, —CH(CH₃)CH₂CH₃), tert-butyl (1-Bu, —C(CH₃)₃), n-pentyl (—CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), n-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, n-heptyl, n-octyl, n-nonyl and n-decyl, etc.

The term “alkoxy” refers to an alkyl group, as previously defined, attached to the parent molecular moiety via an oxygen atom. Unless otherwise specified, the alkoxy group contains 1-12 carbon atoms. In some embodiments, the alkoxy group contains 1-8 carbon atoms; in other embodiments, the alkoxy group contains 1-6 carbon atoms; in still other embodiments, the alkoxy group contains 1~4 carbon atoms; in yet other embodiments, the alkoxy group contains 1-3 carbon atoms. The alkoxy group may be optionally substituted with one or more substituents disclosed herein.

Some non-limiting examples of the alkoxy group include, but are not limited to, methoxy (MeO, —OCH₃), ethoxy (EtO, —OCH₂CH₃), 1-propoxy (n-PrO, n-propoxy, —OCH₂CH₂CH₃), 2-propoxy (i-PrO, i-propoxy, —OCH(CH₃)₂), 1-butoxy (n-BuO, n-butoxy, —OCH₂CH₂CH₂CH₃), 2-methyl-1-propoxy (i-BuO, i-butoxy, —OCH₂CH(CH₃)₂), 2-butoxy (s-BuO, s-butoxy, —OCH(CH₃)CH₂CH₃), 2-methyl-2-propoxy (i-BuO, t-butoxy, —OC(CH₃)₃), 1-pentoxy (n-pentoxy, —OCH₂CH₂CH₂CH₂CH₃), 2-pentoxy (—OCH(CH₃)CH₂CH₂CH₃), 3-pentoxy (—OCH(CH₂CH₃)₂), 2-methyl-2-butoxy (—OC(CH₃)₂CH₂CH₃), 3-methyl-2-butoxy (—OCH(CH₃)CH(CH₃)₂), 3-methyl-1-butoxy (—OCH₂CH₂CH(CH₃)₂), 2-methyl-1-butoxy (—OCH₂CH(CH₃)CH₂CH₃), and the like.

The term “alkenyl” refers to a linear or branched monovalent, divalent or polyvalent hydrocarbon group containing 2-12 carbon atoms, or 2-8 carbon atoms, or 2-6 carbon atoms, or 2-4 carbon atoms, wherein at least one position of the C—C is an sp² double bond, wherein the alkenyl group can be independently unsubstituted or substituted with one or more substituents described in the present invention, including “cis”, “trans” or “Z”, “E” isomers, specific examples of which include, but are not limited to, vinyl (—CH═CH₂), propenyl (—CH═CHCH₃), allyl (—CH₂CH═CH₂), etc., wherein the alkenyl group can be independently unsubstituted or substituted with one or more substituents described in the present invention.

The term “consisting of M-Mi ring atoms” means that the cyclic group is composed of M-Mi ring atoms, and the ring atoms include carbon atoms and/or heteroatoms such as O, N, S and P. For example, “heterocyclyl consisting of 3-6 ring atoms” means a monocyclic heterocyclyl consisting of 3, 4, 5 or 6 ring atoms.

The term “heterocyclyl” refers to a saturated or unsaturation, nonaromatic, monocyclic, bicyclic or tricyclic ring system in which at least one ring member is selected from nitrogen, sulfur or oxygen. Wherein, the heterocyclyl group may be optionally substituted with one or more substituents disclosed herein. Unless otherwise specified, the heterocyclyl group may be carbon or nitrogen linked, and a —CH₂— group can be optionally replaced by —C(═O)— or —C(═S)—. The sulfur can be optionally oxygenized to S-oxide The nitrogen can be optionally oxygenized to N-oxide. In some embodiments, the heterocyclyl is a heterocyclyl consisting of 3-12 ring atoms, in some embodiments, the heterocyclyl is a heterocyclyl consisting of 5-10 ring atoms, in some embodiments, the heterocyclyl is a heterocyclyl consisting of 3-6 ring atoms, in some embodiments, the heterocyclyl is a heterocyclyl consisting of 4-6 ring atoms, in some embodiments, the heterocyclyl is a heterocyclyl consisting of 5-6 ring atoms, in other embodiments, the heterocyclyl is a heterocyclyl consisting of 4 atoms, which refers to a monovalent or polyvalent, saturated or partially unsaturated, non-aromatic monocyclic ring containing 4 ring atoms, wherein at least one ring atom is selected from nitrogen, sulfur and oxygen atoms. In other embodiments, the heterocyclyl is a heterocyclyl consisting of 5 atoms, which refers to a monovalent or multivalent, saturated or partially unsaturated, non-aromatic monocyclic ring containing 5 ring atoms, wherein at least one ring atom is selected from nitrogen, sulfur and oxygen atoms. In other embodiments, the heterocyclyl is a heterocyclyl consisting of 6 atoms, which refers to a monovalent or multivalent, saturated or partially unsaturated, non-aromatic monocyclic ring containing 6 ring atoms, wherein at least one ring atom is selected from nitrogen, sulfur and oxygen atoms. The “heterocyclyl” also includes a group in which the heterocyclyl is fused with a saturated or partially unsaturated ring or a heterocyclic ring. Some non-limiting examples of the heterocyclyl include pyrrolidinyl, tetrahydrofuranyl, dihydrofuranyl, tetrahydrothienyl, tetrahydropyranyl, dihydropyranyl, tetrahydrothiopyranyl, piperidinyl, morpholinyl, thiomorpholinyl, thioxanyl, piperazinyl, homopiperazinyl, azetidinyl, oxetanyl, thietanyl, homopiperidinyl, epoxypropyl, azepanyl, oxepanyl, thiepanyl, oxazepinyl, diazepinyl, thiazepinyl, 2-pyrrolinyl, 3-pyrrolinyl, dihydroindolyl, 2H-pyranyl, 4H-pyranyl, dioxanyl, 1,3-dioxolanyl, pyrazolinyl, dithianyl, dithiolanyl, dihydrothienyl, pyrazolidinyl, imidazolinyl, imidazolidinyl, 1,2,3,4-tetrahydroisoquinolinyl, 3-azabicyclo[3.1.0]hexyl, 3-azabicyclo[4.1.0]heptyl, azabicyclo[2.2.2]hexyl, 3H-indolylquinolizinyl and N-pyridylurea. Examples of heterocyclic groups also include 1,1-dioxothiomorpholinyl, examples wherein the carbon atoms on the ring are substituted with oxo (═O) include, but are not limited to, pyrimidinedionyl, 1,2,4-thiadiazole-5(4H)-onyl, 1,2,4-oxadiazole-5(4H)-onyl, 1H-1,2,4-triazole-5(4H)-onyl, etc., examples wherein the carbon atoms on the ring are substituted with a group ═S include, but are not limited to, 1,2,4-oxadiazole-5(4H)-thionyl, 1,3,4-oxadiazole-2(3H)-thionyl, etc.

The term “heteroatom” refers to one or more of O, S, N, P and Si, including N, S and P in any oxidation state; in the form of primary, secondary, tertiary amines and quaternary ammonium salts; or in the form of a nitrogen atom in a heterocyclic ring in which the hydrogen is substituted, for example, N (as N in 3,4-dihydro-2H-pyrrolyl), NH (as NH in pyrrolidinyl) or NR (as NH in N-substituted pyrrolidinyl), wherein R represents a substituent as described herein.

The terms “alkylsilyl” and “alkylsilyl” refer to silyl (—SiH₃) groups in which the hydrogen atoms are independently replaced by 1, 2 or 3 alkyl groups, respectively, wherein, in some embodiments, the alkylsilyl group is a lower alkylsilyl group formed by 1, 2 or 3 Cr-z alkyl groups attached to a silicon atom. In other embodiments, the alkylsilyl group is a lower alkylsilyl group formed by 1, 2 or 3 C₁₋₉ alkyl groups attached to a silicon atom. In other embodiments, the alkylsilyl group is a lower alkylsilyl group formed by 1, 2 or 3 C₁₋₆ alkyl groups attached to a silicon atom. In other embodiments, the alkylsilyl group is a lower alkylsilyl group formed by 1, 2 or 3 C₁₋₄ alkyl groups attached to a silicon atom. In another embodiments, the alkylsilyl group is a lower alkylsilyl group formed by 1, 2 or 3 C₁₋₃ alkyl groups attached to a silicon atom. Suitable alkylsilyl groups can be monoalkylsilyl, dialkylsilyl or trialkylsilyl. Examples of alkylsilyl groups include, but are not limited to, trimethylsilyl (—Si(CH₃)₃), triethylsilyl (—Si(CH₂CH₃)₃), tri-n-propylsilyl (—Si(CH₂CH₂CH₃)₃), and the like.

The term “hydroxy protecting group” refers to a labile chemical moiety that protects a hydroxy group against undesirable reactions during one or more synthetic procedures. After the one or more synthetic procedures, the hydroxy protecting group can be selectively removed. Hydroxyl protecting groups known in the art are generally described in T. H. Greene and P. G. M. Wuts, Protective Groups in Organic Synthesis, 3rd ed., John Wiley & Sons, New York (1999). Examples of hydroxy protecting groups of the present invention include, but are not limited to, C₁₋₁₂ alkyl, benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropyloxycarbonyl, biphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl (Ac or —C(O)CH₃), formyl, chloroacetyl, trifluoroacetyl, methoxyacetyl, phenoxyacetyl, benzoyl (Bz or —C(O) C₆H₅), C₁₋₁₀ alkyl (methyl, tert-butyl, etc.), 2,2,2-trichloroethyl, 2-trimethylsilylethyl, 1,1-dimethyl-2-propenyl, 3-methyl-3-butenyl, allyl, C₆₋₁₀ aryl C₁₋₄ alkyl (such as benzyl, phenethyl, etc.), p-methoxybenzyldiphenylmethyl, triphenylmethyl (triphenylmethyl or trityl), tetrahydrofuranyl, methoxymethyl, methylthiomethyl, benzyloxymethyl, 2,2,2-trichloroethoxymethyl, 2-(trimethylsilyl)ethoxymethyl, methylsulfonyl, p-toluenesulfonyl, C₁₋₁₀ alkylsilyl (such as trimethylsilyl (TMS or —Si(CH₃)₃)), triethylsilyl, triisopropylsilyl, MMTr, DMTr or 4′,4′,4′-trimethoxytrityl, etc.

The term “amino protecting group” refers to a labile chemical moiety that protects an amino group against undesirable reactions during synthetic procedures. After the one or more synthetic procedures, the amino protecting group can be selectively removed as described herein. Amino protecting groups known in the art are generally described in T. H. Greene and P. G. M. Wuts, Protective Groups in Organic Synthesis, 3rd ed., John Wiley & Sons, New York (1999). Examples of amino protecting groups include, but are not limited to, acetyl, tert-butoxycarbonyl, 9-fluorenylmethoxycarbonyl, benzyloxycarbonyl, and the like.

The term “solid support” specifically denotes any particle, bead or surface on which oligonucleotide synthesis can take place. For example, both inorganic solid supports and organic solid supports can be selected for use in the embodiments of the present invention. The inorganic solid support is preferably selected from silica gel and controlled-pore glass beads (CPG). The organic solid support is a resin, preferably a macroporous resin, more preferably highly cross-linked polystyrene, Tentagel (a graft copolymer composed of a low-cross-linked polystyrene matrix with polyethylene glycol (PEG or POE) grafted thereon), polyvinyl acetate (PVA), Poros-polystyrene/divinylbenzene copolymer, aminopolyethylene glycol and cellulose, etc. Preferred embodiments of the present invention utilize CPG-based solid supports. Many other commercially available solid supports are contemplated by the present invention.

### DESCRIPTION OF THE DRAWINGS

FIG. 1 shows the inhibitory effects of the present compound DAW30227 (i.e., double-stranded siRNA conjugate S146), the control compound DAW30025 (i.e., double-stranded siRNA conjugate S149), and the vehicle control group (normal saline) on HBsAg in an HBV-Tg mouse model experiment. In the figures, SD represents single dose.

FIG. 2 shows the inhibitory effects of the present compound DAW30335 (i.e., double-stranded siRNA conjugate S147), the control compound DAW30025 (i.e., double-stranded siRNA conjugate S149), DAW30336 (i.e., siRNA conjugate S151), and the vehicle control group (normal saline) on HBsAg in an AAV mouse model experiment.

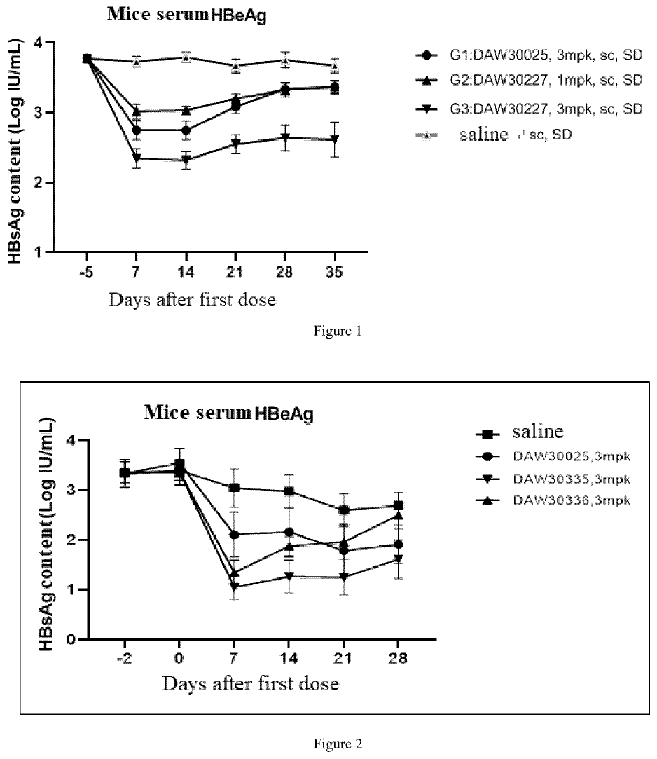

### DETAILED DESCRIPTION OF COMPOUNDS OF THE INVENTION

The present invention provides a new targeting compound, which can be used to prepare a nucleic acid drug for delivering oligonucleotides to the liver in a targeted manner. The present invention also provides a nucleotide conjugate, a preparation method and use thereof, wherein the nucleotide conjugate has higher in vivo delivery efficiency, better stability, higher gene expression inhibition activity in hepatocytes and/or lower toxicity. On the other hand, the present invention further provides an siRNA reagent, a preparation method and application thereof, wherein the siRNA reagent has high in vivo delivery efficiency, good stability, high gene expression inhibition activity in HBV and/or low toxicity.

The present invention aims to provide a novel double-stranded siRNA, a conjugate thereof, a salt thereof and a use thereof, and specifically relates to the use of the double-stranded siRNA and conjugate thereof in preparing a drug for treating and/or preventing hepatitis B. In addition, the present invention also relates to a novel compound, and a use of the compound in raw materials for solid-phase synthesis of DNA nucleotides, raw materials for synthesis of siRNA drugs, siRNA drug research, gene function research and/or screening of whole gene libraries, especially the use of the compound as a raw material for synthesis of the double-stranded siRNA drug described in the present invention. Furthermore, the present invention also relates to a novel nucleotide residue and its use in siRNA drug research, as a raw material for the synthesis of gene function research, and/or as a raw material for screening of whole gene libraries, as well as an embedding group in oligonucleotides. The double-stranded siRNA and conjugate thereof of the present invention have higher gene expression inhibition activity and/or lower toxicity against HBV.

In one aspect, the present invention relates to a double-stranded siRNA, a conjugate thereof, or a salt thereof, the double-stranded siRNA comprises a sense strand and an antisense strand, wherein, the double-stranded siRNA comprises one or more Y (i.e., the double-stranded siRNA of the present

1.-59. (canceled)

60. A double-stranded siRNA, a conjugate or a salt thereof, wherein, the double-stranded siRNA comprises a sense strand and an antisense strand, and the double-stranded siRNA comprises one or more Y, the Y is 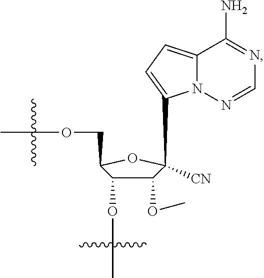 [SMILES: *OC[C@H]1O[C@@](C#N)(c2ccc3c(N)ncnn23)[C@H](OC)[C@@H]1O*]
wherein each of nucleotides in the double-stranded siRNA and Y is independently modified or unmodified;
optionally, the antisense strand comprises a sequence of 5′-UGUGAAGCGAAGUGCACACUU-3′ (SEQ ID NO:40), or a sequence in which one or more nucleotide residues are replaced by Y in the sequence of SEQ ID NO:40; wherein each of nucleotides and Y in the antisense strand is independently modified or unmodified, and the length of the antisense strand is not more than 23 nucleotides;
optionally, the sense strand comprises a sequence of 5′-GUGUGCACUUCGCUUCACA-3′ (SEQ ID NO: 20), or a sequence in which one or more nucleotide residues are replaced by Y in the sequence of SEQ ID NO:20; wherein each of nucleotides and Y in the sense strand is independently modified or unmodified, and the length of the sense strand is not more than 23 nucleotides;
optionally, 70%, 75%, 80%, 85%, 90%, 95% or more of the nucleotides and Y in the sense strand or the antisense strand are modified; optionally, all nucleotides and Y in the double-stranded siRNA are modified.

61. The double-stranded siRNA, conjugate or salt thereof according to claim 60, wherein, the modification is selected from at least one of the following: 2′-methoxy modification, 2′-methoxyethyl modification, 2′-fluoro modification, phosphorothioate linkage, 2′-deoxy modification, 2′-amino modification, locked nucleic acid modification, unlocked nucleic acid modification, ethylene glycol nucleic acid modification and 5′-vinyl phosphate ester modification.

62. The double-stranded siRNA, conjugate or salt thereof according to claim 60, wherein, the sense strand comprises a sequence in which 0, 1, 2, 3, 4 or 5 nucleotide residues are replaced with Y in the sequence of SEQ ID NO:20, wherein each of nucleotides and Y in the sense strand is independently modified or unmodified;
optionally, the antisense strand comprises a sequence in which 0, 1, 2, 3, 4 or 5 nucleotide residues are replaced with Y in the sequence of SEQ ID NO:40, wherein each of nucleotides and Y in the antisense strand is independently modified or unmodified.

63. The double-stranded siRNA, conjugate or salt thereof according to claim 60, wherein, the Y substitution occurs at positions 1 to 19 from the 5′ end of the sequence of SEQ ID NO: 20.
optionally, the Y substitution occurs at positions 1 to 21 from the 5′ end of the sequence of SEQ ID NO: 40.

64. The double-stranded siRNA, conjugate or salt thereof according to claim 60, wherein, the sense strand comprises a sequence in which 0, 1, 2, 3, 4 or 5 nucleotide residues are substituted with Y in the sequence of 5′-g•u•guGcACUucgcuucaca-3′ (SEQ ID NO: 19), wherein each of nucleotides and Y in the sense strand is independently modified or unmodified, and the Y substitution occurs at positions 1 to 19 from the 5′ end of the sequence of SEQ ID NO: 19, and the length of the sense strand is not more than 23 nucleotides;
optionally, the antisense strand comprises a sequence in which 0, 1, 2, 3, 4 or 5 nucleotide residues are substituted with Y in the sequence of 5′-u•G•ugaAgCGaaguGcAcac•u•u-3′ (SEQ ID NO: 36), wherein each of nucleotides and Y in the antisense strand is independently modified or unmodified, and the Y substitution occurs at positions 1 to 21 from the 5′ end of the sequence of SEQ ID NO: 36, and the length of the antisense strand is not more than 23 nucleotides; or
the antisense strand comprises a sequence in which 0, 1, 2, 3, 4 or 5 nucleotide residues are substituted with Y in the sequence of 5′-u•G•uga(Agn)gCGaaguGcAcac•u•u-3′ (SEQ ID NO: 33), wherein each of nucleotides and Y in the antisense strand is independently modified or unmodified, and the Y substitution occurs at positions 1 to 21 from the 5′ end of the sequence of SEQ ID NO: 33, and the length of the antisense strand is not more than 23 nucleotides.

65. The double-stranded siRNA, conjugate or salt thereof according to claim 60, the sense strand comprises one of the following nucleotide sequences:
SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO: 12, SEQ ID NO:13, SEQ ID NO: 14, SEQ ID NO: 15, SEQ ID NO: 16, SEQ ID NO: 17, SEQ ID NO: 18, SEQ ID NO: 19 and SEQ ID NO:21,
the length of the sense strand is not more than 23 nucleotides;
optionally, the antisense strand comprises one of the following nucleotide sequences:
SEQ ID NO:22, SEQ ID NO:23, SEQ ID NO:24, SEQ ID NO:25, SEQ ID NO:26, SEQ ID NO: 27, SEQ ID NO:28, SEQ ID NO:29, SEQ ID NO:30, SEQ ID NO:31, SEQ ID NO:32, SEQ ID NO: 33, SEQ ID NO:34, SEQ ID NO:35, SEQ ID NO:36, SEQ ID NO:37, SEQ ID NO:38 and SEQ ID NO:39,
the length of the sense strand is not more than 23 nucleotides; or
the double-stranded siRNA comprises one of the following:
a sense strand comprising the nucleotide sequence of SEQ ID NO:15 and an antisense strand comprising the nucleotide sequence of SEQ ID NO:25, wherein the sense strand and antisense strand are each independently not more than 23 nucleotides;
a sense strand comprising the nucleotide sequence of SEQ ID NO:17 and an antisense strand comprising the nucleotide sequence of SEQ ID NO:25, wherein the sense strand and antisense strand are each independently not more than 23 nucleotides;
a sense strand comprising the nucleotide sequence of SEQ ID NO: 15 and an antisense strand comprising the nucleotide sequence of SEQ ID NO:26, wherein the sense strand and antisense strand are each independently not more than 23 nucleotides;
a sense strand comprising the nucleotide sequence of SEQ ID NO:16 and an antisense strand comprising the nucleotide sequence of SEQ ID NO:26, wherein the sense strand and antisense strand are each independently not more than 23 nucleotides;
a sense strand comprising the nucleotide sequence of SEQ ID NO:17 and an antisense strand comprising the nucleotide sequence of SEQ ID NO:26, wherein the sense strand and antisense strand are each independently not more than 23 nucleotides;
a sense strand comprising the nucleotide sequence of SEQ ID NO:15 and an antisense strand comprising the nucleotide sequence of SEQ ID NO:27, wherein the sense strand and antisense strand are each independently not more than 23 nucleotides;
a sense strand comprising the nucleotide sequence of SEQ ID NO: 16 and an antisense strand comprising the nucleotide sequence of SEQ ID NO:27, wherein the sense strand and antisense strand are each independently not more than 23 nucleotides;
a sense strand comprising the nucleotide sequence of SEQ ID NO:17 and an antisense strand comprising the nucleotide sequence of SEQ ID NO:27, wherein the sense strand and antisense strand are each independently not more than 23 nucleotides;
a sense strand comprising the nucleotide sequence of SEQ ID NO: 16 and an antisense strand comprising the nucleotide sequence of SEQ ID NO:28, wherein the sense strand and antisense strand are each independently not more than 23 nucleotides;
a sense strand comprising the nucleotide sequence of SEQ ID NO: 17 and an antisense strand comprising the nucleotide sequence of SEQ ID NO:28, wherein the sense strand and antisense strand are each independently not more than 23 nucleotides; or
a sense strand comprising the nucleotide sequence of SEQ ID NO:18 and an antisense strand comprising the nucleotide sequence of SEQ ID NO:28, wherein the sense strand and antisense strand are each independently not more than 23 nucleotides.

66. The double-stranded siRNA, conjugate or salt thereof according to claim 60, wherein, the double-stranded siRNA or conjugate thereof is selected from one of S1 to S143, S146, S147 and S150.

67. The double-stranded siRNA, conjugate or salt thereof according to claim 60, wherein, the double-stranded siRNA conjugate is a conjugate of the double-stranded siRNA conjugated with a conjugation group L96 or DAW40007-4, and the structures of the conjugation groups L-96 and DAW40007-4 are respectively:

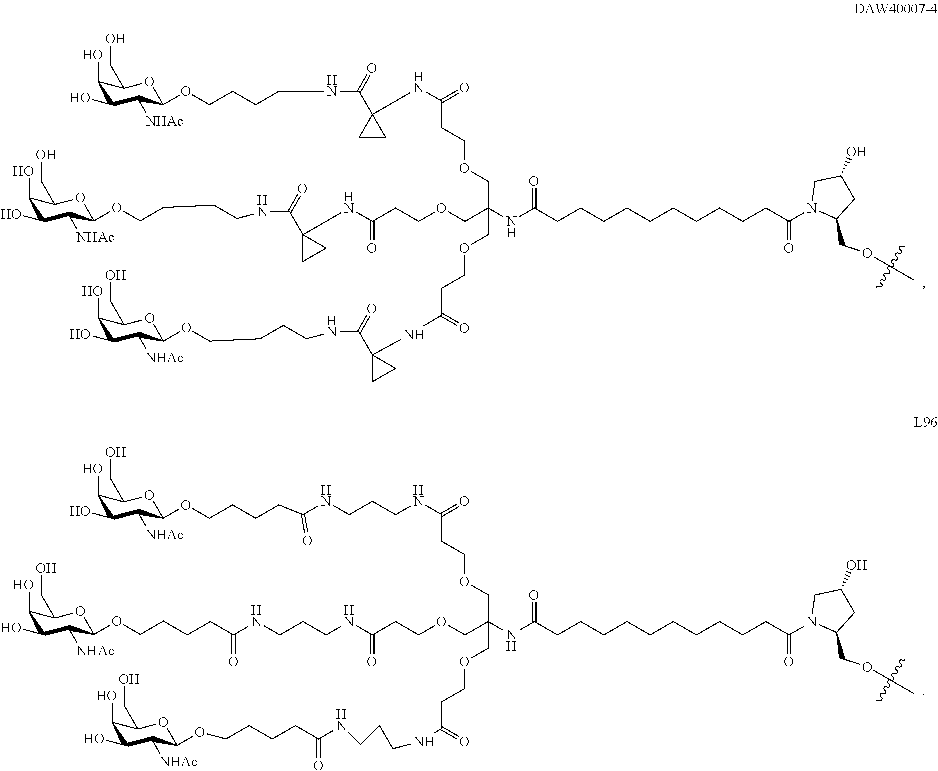

68. The double-stranded siRNA, conjugate or salt thereof according to claim 67, wherein, the conjugated group is conjugated to the 3′ end or the 5′ end of the sense strand of the double-stranded siRNA, preferably to the 3′ end of the sense strand of the double-stranded siRNA, wherein, the double-stranded siRNA is linked to the conjugated group through a phosphate eater group, phosphorothioate group or a phosphate group.

69. A nucleotide residue Y having structure:

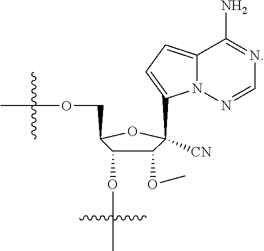 [SMILES: *OC[C@H]1O[C@@](C#N)(c2ccc3c(N)ncnn23)[C@H](OC)[C@@H]1O*]

70. A compound having Formula (I) or a stereoisomer, a tautomer or an acceptable salt of the compound having Formula (I), 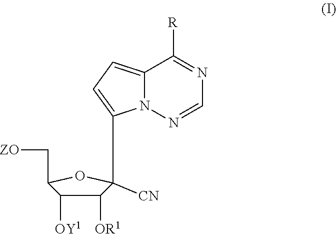
R¹ is methyl, ethyl, n-propyl, i-propyl, n-butyl, i-butyl or tert-butyl, each of the methyl, ethyl, n-propyl, i-propyl, n-butyl, i-butyl or tert-butyl is independently optionally substituted with 1, 2, 3 or 4 substituents selected from deuterium, hydroxyl, cyano, F, Cl, Br, I, methoxy, ethoxy, 1-propoxy and 2-propoxy;
Z and Y¹ are each independently H, deuterium, a hydroxyl protecting group, HOC(═O)(CH₂)ⱼC(═O)—, M-C(═O)(CH₂)ⱼC(═O)—, a phosphate ester group, a phosphorothioate group, a phosphoramidite group or a hydrogen phosphate group;
M is a solid support, preferably a hydroxyl- or amino-functionalized solid support, more preferably a resin or CPG, further preferably a macroporous resin or CPG, further preferably an aminomethyl resin, a hydroxyl resin or —NHCPG;
R is —NHR² or —N—CH—NR^{a}R^{b};
R² is an amino protecting group;
each of R^{a} and R^{b} is independently H or an amino protecting group, or R^{a}, R^{b} and the nitrogen atom to which they are attached form a heterocyclic group consisting of 5-6 ring atoms, each of heterocyclic group consisting of 5-6 ring atoms is independently optionally substituted with 1, 2, 3 or 4 substituents selected from deuterium, hydroxyl, cyano, F, Cl, Br, I, methyl, ethyl, n-propyl, isopropyl, methoxy, ethoxy, 1-propoxy and 2-propoxy;
wherein, each j is independently 1, 2, 3, 4 or 5.

71. The compound according to claim 70, wherein, Z and Y¹ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₁₂ alkyl, C₁₋₁₂ alkylC(═O)—, C₁₋₁₂ alkylsilyl, C₆₋₁₀ arylC₁₋₆ alkyl, HOC(═O)(CH₂)ⱼC(═O)—, M-C(═O)(CH₂)ⱼC(═O)—, triphenylmethyl, MMTr, DMTr, 4′,4′,4′-trimethoxytrityl, 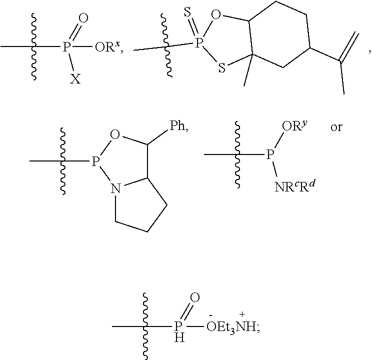
or
Z and Y¹ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₁₀ alkyl, C₁₋₁₀ alkylC(═O)—, C₁₋₁₀ alkylsilyl, C₆₋₁₀ arylC₁₋₄ alkyl, HOC(═O)(CH₂)ⱼC(═O)—, M-C(═O)(CH₂)ⱼC(═O)—, triphenylmethyl, MMTr, DMTr, 4′,4′,4′-trimethoxytrityl, 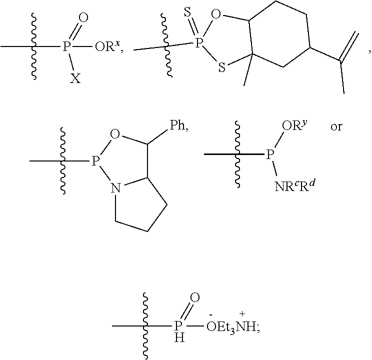
or
Z and Y¹ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₈ alkyl, C₁₋₈ alkylC(═O)—, C₁₋₈ alkylsilyl, phenylC₁₋₃ alkyl, HOC(═O)(CH₂)ⱼC(═O)—, M-C(═O)(CH₂)ⱼC(═O)—, triphenylmethyl, MMTr, DMTr, 4′,4′,4′-trimethoxytrityl, 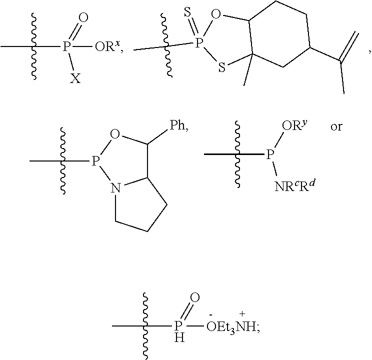
or
Z and Y¹ are each independently H, deuterium, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, C₁₋₆ alkyl, C₁₋₆ alkylC (—O)—, C₁₋₆ alkylsilyl, benzyl, phenylethyl, HOC(═O)(CH₂)ⱼC(═O)—, M-C(═O)(CH₂)ⱼC(═O)—, triphenylmethyl, MMTr, DMTr, 4′,4′,4′-trimethoxytrityl, 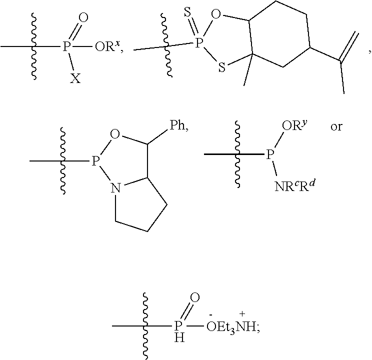
wherein, X is Cl or Br;
each of R^{x} and R^{y} is independently a hydroxy protecting group; or each of R^{x} and R^{y} is independently benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, cyano C₁₋₆ alkyl, C₁₋₆ alkyl, C₁₋₆ alkyl C(═O)—, C₁₋₆ alkylsilyl, phenyl, halogen-substituted phenyl, benzyl or phenethyl; or
each of R^{x} and R^{y} is independently benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2,2,2-trichloroethyl, 2-trimethylsilylethyl, 1,1-dimethyl-2-propenyl, 3-methyl-3-butenyl, allyl, p-methoxybenzyldiphenylmethyl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, CNCH₂—, CN(CH₂)₂—, CNCH(CH₃)₂CH₂—, CN(CH₂)₃—, CNCH(CH₃)₂CH₂CH₂—, methyl, ethyl, n-propyl, i-propyl, n-butyl, i-butyl, tert-butyl, C₁₋₄ alkyl C(═O)—, C₁₋₄ alkylsilyl, phenyl, halogen-substituted phenyl, benzyl or phenethyl;
each of R^{c} and R^{d} is independently H or an amino protecting group; or R^{c}, R^{d} and the nitrogen atom to which they are attached form a heterocyclic group consisting of 5-6 ring atoms, the heterocyclic group consisting of 5-6 ring atoms is independently optionally substituted with 1, 2, 3 or 4 substituents selected from deuterium, hydroxyl, cyano, F, Cl, Br, I, methyl, ethyl, propyl, methoxy, ethoxy, 1-propoxy and 2-propoxy; or each of R^{c} and R^{d} is independently H, methyl, ethyl, n-propyl, i-propyl, n-butyl, i-butyl, tert-butyl, methoxy, ethoxy, 1-propoxy, 2-propoxy, 9-fluorenylmethoxycarbonyl, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2-trimethylsilylethyl, 1,1-dimethyl-2-propenyl, 3-methyl-3-butenyl, allyl, benzyl, p-methoxybenzyldiphenylmethyl, trityl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, trimethylsilyl, triethylsilyl, triisopropylsilyl or R⁴C(═O)—, or R^{c}, R^{d} and the N atom to which they are attached form a pyrrolidinyl, morpholinyl, piperidinyl or piperazinyl, each of pyrrolidinyl, morpholinyl, piperidinyl and piperazinyl is independently optionally substituted with 1, 2, 3 or 4 substituents selected from deuterium, hydroxyl, cyano, F, Cl, Br, I, methoxy, ethoxy, 1-propoxy and 2-propoxy.

72. The compound according to claim 70, wherein, R² is 9-fluorenylmethoxycarbonyl, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, methyl, tert-butyl, 2-trimethylsilylethyl, 1,1-dimethyl-2-propenyl, 3-methyl-3-butenyl, allyl, benzyl, p-methoxybenzyldiphenylmethyl, trityl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, trimethylsilyl, triethylsilyl, triisopropylsilyl, C₁₋₆ alkoxy or R³C(═O)—;
each of R^{a} and R^{b} is independently H, methyl, ethyl, n-propyl, i-propyl, n-butyl, i-butyl, tert-butyl, methoxy, ethoxy, 1-propoxy, 2-propoxy, 9-fluorenylmethoxycarbonyl, benzyloxycarbonyl, 4-nitrobenzyloxycarbonyl, 4-bromobenzyloxycarbonyl, 4-methoxybenzyloxycarbonyl, biphenylmethoxycarbonyl, 2,2,2-trichloroethoxycarbonyl, 2-(trimethylsilyl)ethoxycarbonyl, allyloxycarbonyl, chloroacetyl, trifluoroacetyl, methoxyacetyl, phenoxyacetyl, benzoyl, 2-trimethylsilylethyl, 1,1-dimethyl-2-propenyl, 3-methyl-3-butenyl, allyl, benzyl, p-methoxybenzyldiphenylmethyl, trityl, tetrahydrofuranyl, methoxymethyl, methylthiomethyl, benzyloxymethyl, 2,2,2-trichloroethoxymethyl, 2-(trimethylsilyl)ethoxymethyl, methanesulfonyl, p-toluenesulfonyl, trimethylsilyl, triethylsilyl, triisopropylsilyl or R⁴C(═O)—, or R^{a}, R^{b} and the N atom to which they are attached form a pyrrolidinyl, morpholinyl, piperidinyl or piperazinyl, each of pyrrolidinyl, morpholinyl, piperidinyl and piperazinyl is independently optionally substituted with 1, 2, 3 or 4 substituents selected from deuterium, hydroxyl, cyano, F, Cl, Br, I, methoxy, ethoxy, 1-propoxy and 2-propoxy;
wherein, each of R³ and R⁴ is independently C₁₋₆ alkyl, halo C₁₋₆ alkyl, C₁₋₆ alkoxy, phenyl, halo-substituted phenyl, C₁₋₆ alkylphenyl or benzyl; or
each of R³ and R⁴ is independently C₁₋₄ alkyl, halo C₁₋₄ alkyl, C₁₋₄ alkoxy, phenyl, halo-substituted phenyl, C₁₋₄ alkylphenyl or benzyl; or
each of R³ and R⁴ is independently methyl, ethyl, n-propyl, isopropyl, trifluoromethyl, difluoromethyl, monofluoromethyl, trichloromethyl, dichloromethyl, monochloromethyl, 2,2,2-trichloroethyl, methoxy, ethoxy, 1-propoxy, 2-propoxy, 1-butoxy, 2-methyl-1-propoxy, 2-butoxy, tert-butoxy, phenyl, halogen-substituted phenyl, C₁₋₃ alkylphenyl or benzyl.

73. The compound according to claim 70 having a structure as shown in formula (II),

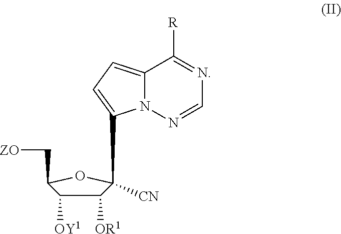

74. A pharmaceutical composition comprising the double-stranded siRNA, conjugate or salt thereof according to claim 60, and a pharmaceutically acceptable carrier.

75. A method for treating and/or preventing hepatitis B, comprising using the double-stranded siRNA conjugate, conjugate and salt thereof according to claim 60.

76. A method for treating and/or preventing hepatitis B, comprising using the pharmaceutical composition according to claim 74.

77. A method for solid-phase synthesis of DNA nucleotides, synthesis of oligonucleotide drugs, synthesis of siRNA drugs, siRNA drug research, gene function research and/or screening of a whole gene library, the method comprises using the compound according to claim 70.

78. A method for inhibiting or blocking gene expression, comprising using a nucleotide sequence comprising 10 to 50 nucleotides or nucleotide base pairs, wherein the nucleotide sequence has embedded the nucleotide residues according to claim 69, wherein, the gene is an HBV gene;
wherein, the nucleotide sequence is siRNA; wherein, the siRNA comprises the sense strand and the antisense strand; wherein, the nucleotide residues are embedded only into the sense strand of the siRNA; or the nucleotide residues are embedded only into the antisense strand of the siRNA; or
the nucleotide residues are embedded into the sense strand and the antisense strand of the siRNA.

79. The method according to claim 78, wherein, the sense strand of the siRNA comprises the sequence of SEQ ID NO:19;
the antisense strand of the siRNA comprises the sequence of SEQ ID NO:36 or SEQ ID NO: 33.


SEQUENCE LISTING (46 sequences)

SEQ ID NO: 1 - RNA, 19 nt, Albies alba
  source 1..19: mol_type = other RNA
gygtgcacttcgcttcaca

SEQ ID NO: 2 - RNA, 19 nt, Albies alba
  source 1..19: mol_type = other RNA
gtytgcacttcgcttcaca

SEQ ID NO: 3 - RNA, 19 nt, Albies alba
  source 1..19: mol_type = other RNA
gtgygcacttcgcttcaca

SEQ ID NO: 4 - RNA, 19 nt, Albies alba
  source 1..19: mol_type = other RNA
gtgtycacttcgcttcaca

SEQ ID NO: 5 - RNA, 19 nt, Albies alba
  source 1..19: mol_type = other RNA
gtgtgyacttcgcttcaca

SEQ ID NO: 6 - RNA, 19 nt, Albies alba
  source 1..19: mol_type = other RNA
gtgtgcycttcgcttcaca

SEQ ID NO: 7 - RNA, 19 nt, Albies alba
  source 1..19: mol_type = other RNA
gtgtgcayttcgcttcaca

SEQ ID NO: 8 - RNA, 19 nt, Albies alba
  source 1..19: mol_type = other RNA
gtgtgcacytcgcttcaca

SEQ ID NO: 9 - RNA, 19 nt, Albies alba
  source 1..19: mol_type = other RNA
gtgtgcactycgcttcaca

SEQ ID NO: 10 - RNA, 19 nt, Albies alba
  source 1..19: mol_type = other RNA
gtgtgcacttygcttcaca

SEQ ID NO: 11 - RNA, 19 nt, Albies alba
  source 1..19: mol_type = other RNA
gtgtgcacttcycttcaca

SEQ ID NO: 12 - RNA, 19 nt, Albies alba
  source 1..19: mol_type = other RNA
gtgtgcacttcgyttcaca

SEQ ID NO: 13 - RNA, 19 nt, Albies alba
  source 1..19: mol_type = other RNA
gtgtgcacttcgcytcaca

SEQ ID NO: 14 - RNA, 19 nt, Albies alba
  source 1..19: mol_type = other RNA
gtgtgcacttcgctycaca

SEQ ID NO: 15 - RNA, 19 nt, Albies alba
  source 1..19: mol_type = other RNA
gtgtgcacttcgcttyaca

SEQ ID NO: 16 - RNA, 19 nt, Albies alba
  source 1..19: mol_type = other RNA
gtgtgcacttcgcttcyca

SEQ ID NO: 17 - RNA, 19 nt, Albies alba
  source 1..19: mol_type = other RNA
gtgtgcacttcgcttcaya

SEQ ID NO: 18 - RNA, 19 nt, Albies alba
  source 1..19: mol_type = other RNA
gtgtgcacttcgcttcacy

SEQ ID NO: 19 - RNA, 19 nt, Albies alba
  source 1..19: mol_type = other RNA
gtgtgcacttcgcttcaca

SEQ ID NO: 20 - RNA, 19 nt, Albies alba
  source 1..19: mol_type = other RNA
gtgtgcacttcgcttcaca

SEQ ID NO: 21 - RNA, 19 nt, Albies alba
  source 1..19: mol_type = other RNA
gtgtgcacttcgcttcyca

SEQ ID NO: 22 - RNA, 21 nt, Albies alba
  source 1..21: mol_type = other RNA
tytgaagcgaagtgcacactt

SEQ ID NO: 23 - RNA, 21 nt, Albies alba
  source 1..21: mol_type = other RNA
tgygaagcgaagtgcacactt

SEQ ID NO: 24 - RNA, 21 nt, Albies alba
  source 1..21: mol_type = other RNA
tgtyaagcgaagtgcacactt

SEQ ID NO: 25 - RNA, 21 nt, Albies alba
  source 1..21: mol_type = other RNA
tgtgyagcgaagtgcacactt

SEQ ID NO: 26 - RNA, 21 nt, Albies alba
  source 1..21: mol_type = other RNA
tgtgaygcgaagtgcacactt

SEQ ID NO: 27 - RNA, 21 nt, Albies alba
  source 1..21: mol_type = other RNA
tgtgaaycgaagtgcacactt

SEQ ID NO: 28 - RNA, 21 nt, Albies alba
  source 1..21: mol_type = other RNA
tgtgaagygaagtgcacactt

SEQ ID NO: 29 - RNA, 21 nt, Albies alba
  source 1..21: mol_type = other RNA
tgtgaagcgyagtgcacactt

SEQ ID NO: 30 - RNA, 21 nt, Albies alba
  source 1..21: mol_type = other RNA
tgtgaagcgaygtgcacactt

SEQ ID NO: 31 - RNA, 21 nt, Albies alba
  source 1..21: mol_type = other RNA
tgtgaagcgaagtgcycactt

SEQ ID NO: 32 - RNA, 21 nt, Albies alba
  source 1..21: mol_type = other RNA
tgtgaagcgaagtgcacyctt

SEQ ID NO: 33 - RNA, 21 nt, Albies alba
  source 1..21: mol_type = other RNA
tgtgaagcgaagtgcacactt

SEQ ID NO: 34 - RNA, 21 nt, Albies alba
  source 1..21: mol_type = other RNA
tgtgaagcgaagtgcycactt

SEQ ID NO: 35 - RNA, 21 nt, Albies alba
  source 1..21: mol_type = other RNA
tgtgaagcgaagtgcacyctt

SEQ ID NO: 36 - RNA, 21 nt, Albies alba
  source 1..21: mol_type = other RNA
tgtgaagcgaagtgcacactt

SEQ ID NO: 37 - RNA, 21 nt, Albies alba
  source 1..21: mol_type = other RNA
tgtgayycgaagtgcacactt

SEQ ID NO: 38 - RNA, 21 nt, Albies alba
  source 1..21: mol_type = other RNA
tgtgaagcgyagtgcacactt

SEQ ID NO: 39 - RNA, 21 nt, Albies alba
  source 1..21: mol_type = other RNA
tgtgaagcgaygtgcacactt

SEQ ID NO: 40 - RNA, 21 nt, Albies alba
  source 1..21: mol_type = other RNA
tgtgaagcgaagtgcacactt

SEQ ID NO: 41 - RNA, 19 nt, Albies alba
  source 1..19: mol_type = other RNA
gtgtgcacttcgcttyaca

SEQ ID NO: 42 - RNA, 19 nt, Albies alba
  source 1..19: mol_type = other RNA
gtgtgcacttcgcttcaca

SEQ ID NO: 43 - RNA, 19 nt, Albies alba
  source 1..19: mol_type = other RNA
gtgtgcacttcgcttcaca

SEQ ID NO: 44 - RNA, 19 nt, Albies alba
  source 1..19: mol_type = other RNA
gtgtgcacttcgcttcaya

SEQ ID NO: 45 - RNA, 19 nt, Albies alba
  source 1..19: mol_type = other RNA
gtgtgcacttcgcttcaca

SEQ ID NO: 46 - RNA, 21 nt, Albies alba
  source 1..21: mol_type = other RNA
tgtgaagcgaagtgcacactt